(19) United States
(12) Patent Application Publication
(10) Pub. No.: US 2012/0213837 A1
(43) Pub. Date: Aug. 23, 2012

(54) COMPOSITIONS AND METHODS FOR BIOACTIVE COATINGS TO IMPROVE ALLOGRAFT INCORPORATION

(75) Inventor: Edward A. Botchwey, III, Charlottesville, VA (US)

(73) Assignee: UNIVERSITY OF VIRGINIA PATENT FOUNDATION, Charlottesville, VA (US)

(21) Appl. No.: 13/263,812

(22) PCT Filed: Apr. 9, 2010
(86) PCT No.: PCT/US2010/030503

§ 371 Date: May 9, 2012

Related U.S. Application Data
(60) Provisional application No. 61/168,114, filed on Apr. 9, 2009.

(51) Int. Cl. A61K 31/137; A61K 9/00; A61K 31/661; A61P 19/00; A61P 17/02

(52) U.S. Cl. 424/423; 514/653; 514/114

(57) ABSTRACT

The invention provides compositions and methods useful for treating wounds and enhancing wound healing. The present invention discloses a continuous polymer coating system to provide sustained localized delivery of bioactive agents. The data demonstrate the efficacy of a bioactive coating comprising the polymer PLAGA and the agent FTY720, a selective agonist for sphingosine 1-phosphate receptors, and biologically active derivatives and analogs thereof, for use in wound healing. In vitro drug release studies validated 64% loading efficiency with complete release of compound following 14 days. Mechanical evaluation of healing bone showed significant enhancement of mechanical stability in FTY720 treatment groups. Superior osseous integration across the host-graft interface, significant enhancement in smooth muscle cell investment, and reduction in leukocyte recruitment were evident in FTY720 treated groups. The present invention is useful for enhancing angiogenesis for wound healing.

### CROSS REFERENCE TO RELATED APPLICATIONS

This application claims priority under 35 U.S.C. §119(e) to U.S. Provisional Application Ser. No. 61/168,114 filed Apr. 9, 2009, the disclosure of which is incorporated by reference in its entirety herein.

### STATEMENT REGARDING FEDERALLY SPONSORED RESEARCH OR DEVELOPMENT

This invention was made in part with United States Government support under Grant Nos. K01AR052352-01A1, R01AR056445-01A2, and R01DE019935-01 awarded by the NIH. The United States Government has certain rights in the invention.

### BACKGROUND

Bone Grafts

Each year, nearly one million bone graft procedures are performed annually; including 800,000 bone allograft procedures in the United States alone [1, 2]. However, 30%-60% of allograft implants exhibit complications by the 10-year mark. Particularly challenging is the incorporation of massive structural allografts that are commonly used for limb salvage, after tumor resection, and acute trauma. These allografts can provide vastly superior mechanical stability relative to morselized or demineralized allografts, but significant limitations in long-term functional capacity and poor host integration remain, including non-union fractures (10-30%), persistent infection (6-13%), and secondary fractures (10-30%) [3-5]. Notably, mode of fixation to enhance mechanical stability has no influence on rate of complications [6] and attempts to improve overall bone mass to protect against fatigue-induced fracture by local delivery of bone morphogenetic protein 2 (BMP-2) have also failed to reduce long-term complications [7]. Interestingly, most cases presenting post-operative complications including non-union or fracture showed poor revascularization contiguous to the region of graft failure [8, 9], and growing evidence suggests that the largest barrier to successful allograft incorporation and sustained mechanical integrity is not osseous remodeling but delayed or absent vascularization.

Critical size bone defects will not heal during the lifetime of a patient under normal physiologic reactions to bone fracture. Therefore, surgical intervention is necessary and commonly includes the implantation of bone autografts or allografts to treat the bone defect. An ideal bone graft facilitates the processes of bone formation, specifically osteogenesis, osteoinduction, and osteoconduction. Autografts demonstrate all of these properties and their implantation carries no risks of viral transmission. However, autografts exist in limited supply, and the surgical operation to harvest the autograft often results in donor-site morbidity. Bone allografts provide a viable alternative, as they are readily available and avoid the effect of donor-site morbidity. However, allograft tissue is less osteoinductive compared to autografts, and the treated tissue does not contain osteogenic cells. Morselized allograft tissue and demineralized allograft tissue have demonstrated faster incorporation with host bone compared to intact large allograft bone, but the morselized and demineralized allografts do not provide the biomechanical strength of massive allografts. Slow vascularization at the defect site is associated with poor allograft incorporation, particularly for massive structural allografts.

Strong evidence shows that failure of massive structural allografts often occurs because the osteoblast progenitor cells and vessels from the host bone incorporate into the allograft sample very slowly. Thus, utilizing growth factors and molecules to increase the incorporation of osteoprogenitor cells and blood vessels near the defect will presumably enhance the allograft incorporation into host bone and the long-term success of allograft implants.

Angiogenesis

Orthopaedic regenerative medicine has focused on remodeling the microvascular network to prevent ischemia and aid in nutrient and oxygen delivery to sites of injury. An important process which has held great attention in the biomedical arena is angiogenesis. Angiogenesis refers to the growth of new blood vessels, specifically the sprouting of new capillaries from pre-existing vessels which produce new capillary networks. More than four billion dollars have been invested in research and development for angiogenesis based-medicines, establishing this field of study as one of the most heavily funded in history. Additionally, approximately 314 million patients in Western nations can benefit from angiogenesis-stimulating therapies. Hence, it is essential to understand this process and components involved.

In the initial stage of angiogenesis, diseased or injured tissues produce and release growth factors which diffuse into tissues within close proximity. Some of these factors include vascular endothelial growth factor (VEGF), platelet-derived growth factor (PDGF), epidermal growth factor, granulocyte colony-stimulating factor, hepatocyte growth factor, transforming growth factor alpha, and several others. These proteins then bind to and activate specific receptors on endothelial cells. Upon activation, signal pathways are initiated in the endothelial cells which facilitate the production of enzymes. These enzymes create dissolved holes in the basement membrane of existing blood vessels. Endothelial cells then begin to proliferate and subsequently migrate via the dissolved holes of the blood vessels. Next, adhesion molecules, or integrins (αvβ3, αvβ5), facilitate the pulling of new blood vessel sprouts forward. Additional enzymes, called matrix metalloproteinases (MMPs), are created to dissolve the tissue in front of the sprouting vessel tip. These MMPs ensure that as the vessel extends, the tissue is remodeled around the vessel. Blood vessel tubes then begin to form due to sprouting endothelial cells. Once formed, these individual tubes connect to existing blood vessels to create blood vessel loops which can circulate blood. To ensure these newly formed blood vessel tubes are stabilized and functional, smooth muscle cells and pericytes are recruited and provide structural support, essentially allowing blood flow to occur.

Three different processes may contribute to the growth of new blood vessels: vasculogenesis, arteriogenesis, and angiogenesis. Vasculogenesis is the primary process responsible for growth of new vasculature during embryonic development and may play a yet-undefined role in mature adult tissues. It is characterized by differentiation of pluripotent endothelial cell precursors (hemangioblasts or similar cells) into endothelial cells that go on to form primitive blood vessels. Subsequent recruitment of other vascular cell types completes the process of vessel formation. The occurrence of vasculogenesis in mature organisms remains an unsettled issue. It is thought to be unlikely that this process contributes substantially to the new vessel development that occurs spontaneously in response to ischemia or inflammation as a response to growth factor stimulation.

Arteriogenesis refers to the appearance of new arteries possessing a fully developed tunica media. The process may involve maturation of pre-existing collaterals or may reflect de novo formation of mature vessels. Examples of arteriogenesis include formation of angiographically visible collaterals in patients with advanced obstructive coronary or peripheral vascular disease. All vascular cell types, including smooth muscle cells and pericytes, are involved. Arteriogenesis is the preferred type of neovascularization for purposes of restoring myocardial perfusion. Native arterial collateralization is a complex process that involves multiple levels of stimulators, inhibitors, and modulators. Therefore, the discovery of a drug molecule that induces therapeutic arteriogenesis, including the self-propagating cascade of proliferation, migration, and chemotaxis would be useful.

Angiogenesis is the process responsible for formation of new vessels lacking developed media. Examples of angiogenesis include capillary proliferation in wound healing or along the border of myocardial infarction. Angiogenesis can be stimulated by a number of growth factors including fibroblast growth factor-2 (FGF-2) and vascular endothelial growth factor (VEGF). Further, insulin-like growth factor-I (IGF-I) can stimulate proliferation of these cells and can induce VEGF secretion. These growth factors appear to exert their effort directly on endothelial cells and reports indicate that these effects may be mediated through specific integrin molecules (αvβ3 or αvβ5).

The occurrence of both angiogenesis and arteriogenesis has been demonstrated conclusively in a variety of animal models, as well as in patients with coronary disease. Thus, insufficient angiogenesis may lead to tissue ischemia and failure. The recent discovery of novel angiogenic molecules has initiated efforts to improve tissue perfusion via therapeutic angiogenesis. However, rational design of novel treatment strategies and potential drugs mandates a better understanding of the molecular mechanisms of angiogenesis.

S1P-Receptor Targeted Drugs

S1P is an autocrine and paracrine signaling small molecule that impacts proliferation, survival and migration of endothelial cells, mural cells (i.e. vascular smooth muscle cells and pericytes), osteoblasts, and osteoblastic precursors through a family of high-affinity G protein-coupled receptors (S1P1-5) [10-14]. Selectively targeting a subset of S1P receptors with agonists and antagonist compounds (with longer bioactive half-lives than native S1P in vivo), one can control different biological responses. For example, recent reports suggest selective activation of S1P1 and S1P3 receptors via a synthetic analog of S1P, FTY720, promotes the recirculation of osteoclast precursor monocytes from the bone surface, an effect that ameliorates bone loss in models of postmenopausal osteoporosis [15]. Furthermore, FTY720 treatment demonstrates enhanced CXCR4-mediated migration of endothelial progenitor cells and homing of bone marrow progenitors in hindlimb ischemia models [16]. Recent discoveries of smooth muscle cell phenotype regulation in large arteries suggest possible synergies between S1P1 and S1P3, both targets of FTY720. Specifically, daily injections of S1P1/S1P3 antagonist (VPC44116) significantly decreased smooth muscle proliferation and migration [17]. Thus, FTY720 as a single bioactive factor has multiple cellular targets making it an attractive molecule for strategies to improve graft-host integration where multiple biological processes can be simultaneously augmented to address a central limitation, poor vascularization.

It has been shown that sustained release of FTY720 from two-dimensional biodegradable films (1:200 wt/wt) of 50:50 poly-lactic-co-glycolic acid (PLAGA) in the mouse dorsal skinfold window chamber promotes formation of new arterioles and structural enlargement of existing arterioles [18]. This pattern of FTY720-induced microvascular remodeling increases the number and diameter of microvessels, a therapeutic response that is critical for successful integration of allograft implants in vivo. In addition, implantation of 3D PLAGA scaffolds delivering FTY720 to critical size calvarial bone defects significantly increases osseous tissue ingrowth and the proportion of mature smooth muscle cell-invested microvessels within the bony defect [19].

The G-protein coupled signaling pathway of S1P receptors has been shown to enhance cell motility, proliferation, and survival due to S1P stimulation. S1P is secreted by several types of cells including mast cells, macrophages, platelets, and endothelial cells into the blood flow in nanomolar plasma concentrations. In areas of endothelial injury, a higher concentration of S1P is released by activated platelets to aid in wound healing. Thus, S1P is thought to possess significant angiogenic and arteriogenic properties including mural cell recruitment to newly-formed vessels and stimulation of SMC differentiation, proliferation, and migration. S1P also reduces oxygen and nutrient-deprived cell death.

Fingolimod (FTY720) is a synthetic compound that acts as an agonist of the S1P1, S1P3, S1P4, and S1P5 receptors when phosphorylated into FTY720P. Due to its structural similarity with S1P, FTY720 shares many of the effects of natural S1P and thus acts as S1P analog. FTY720 was shown to profoundly stimulate the angiogenic activity and neovascularization of cultured cells. Other studies have shown that FTY720 prolongs allograft survival by preventing perivascular inflammation associated with chronic transplant rejection. Additionally, due to FTY720's rapid initial adsorption and exceptionally long half-life of approximately 7 days, the blood concentration of FTY720 remains relatively stable after administration. Native S1P, on the other hand, is insoluble in aqueous solutions in the absence of a carrier protein and its half-life in blood is less than 1 hour. Therefore, FTY720 may be a more potent therapeutic agent than S1P.

Sphingosine-1-phosphate (S1P) has been demonstrated to induce many cellular effects, including those that result in platelet aggregation, cell proliferation, cell morphology, tumor-cell invasion, endothelial cell chemotaxis and endothelial cell in vitro angiogenesis. For these reasons, S1P receptors are good targets for therapeutic applications such as wound healing and tumor growth inhibition. Sphingosine-1-phosphate signals cells in part via a set of G protein-coupled receptors named S1P1, S1P2, S1P3, S1P4, and S1P5 (formerly Edg-1, Edg-5, Edg-3, Edg-6, and Edg-8, respectively). These receptors share 50-55% identical amino acids and cluster with three other receptors (LPA1, LPA2, and LPA3 (formerly Edg-2, Edg-4 and Edg-7)) for the structurally related lysophosphatidic acid (LPA).

A conformational shift is induced in the G-Protein Coupled Receptor (GPCR) when the ligand binds to that receptor, causing GDP to be replaced by GTP on the α-subunit of the associated G-proteins and subsequent release of the G-proteins into the cytoplasm. The α-subunit then dissociates from the βγ-subunit and each subunit can then associate with effector proteins, which activate second messengers leading to a cellular response. Eventually the GTP on the G-proteins is hydrolyzed to GDP and the subunits of the G-proteins reassociate with each other and then with the receptor. Amplification plays a major role in the general GPCR pathway. The binding of one ligand to one receptor leads to the activation of many G-proteins, each capable of associating with many effector proteins leading to an amplified cellular response.

S1P receptors make good drug targets because individual receptors are both tissue and response specific. Tissue specificity of the S1P receptors is desirable because development of an agonist or antagonist selective for one receptor localizes the cellular response to tissues containing that receptor, limiting unwanted side effects. Response specificity of the S1P receptors is also of importance because it allows for the development of agonists or antagonists that initiate or suppress certain cellular responses without affecting other responses. For example, the response specificity of the S1P receptors could allow for an S1P mimetic that initiates platelet aggregation without affecting cell morphology.

Sphingosine-1-phosphate is formed as a metabolite of sphingosine in its reaction with sphingosine kinase and is stored in abundance in the aggregates of platelets where high levels of sphingosine kinase exist and sphingosine lyase is lacking S1P is released during platelet aggregation, accumulates in serum, and is also found in malignant ascites. Biodegradation of S1P most likely proceeds via hydrolysis by ectophosphohydrolases, specifically the sphingosine 1-phosphate phosphohydrolases.

The physiologic implications of stimulating individual S1P receptors are largely unknown due in part to a lack of receptor type selective ligands. Isolation and characterization of S1P analogs that have potent agonist or antagonist activity for S1P receptors has been limited due to the complication of synthesis derived from the lack of solubility of S1P analogs.

Polymers

Poly(D,L-lactic-co-glycolic acid) (PLAGA) and poly(3-hydroxybutrate-co-3-hydroxyvalerate) (PHBV) are biodegradable and biocompatible polymers commonly used for tissue-engineered scaffolds (FIG. 1). One can tailor the degradation rate of these polymers by altering the ratio of each component in the polymer composition, thereby rendering them suitable drug-release devices for both local and systemic delivery.

PLAGA is an FDA-approved copolymer of polylactide (PLA) and polyglycolide (PGA). PLA is a hydrophobic material with a degradation time greater than 24 months, which allows for great drug delivery potential. Through metabolic pathways, PLA degrades to lactic acid. PGA is a hydrophilic material and degrades at a faster rate, typically between 6 and 12 months, resulting in the glycolic acid byproduct. The polyester PLAGA degrades through hydrolysis and exhibits bulk degradation, releasing the non-toxic byproducts lactic acid and glycolic acid. Because of these acidic byproducts, local pH changes must be considered during PLAGA degradation. When used as a drug-delivery vehicle, variables such as molecular weight (Mw), copolymer composition, and crystallinity influence polymer degradation and the corresponding drug release kinetics.

PHBV is a polyester copolymer of hydroxybutyrate and hydroxyvalerate with adjustable processing and mechanical properties. By altering the copolymer composition and Mw, one can modify properties of PHBV, such glass transition temperature, crystallinity, and the rate of degradation. The accumulation of degradation products β-hydroxybutyric acid and hydroxyvaleric acid can thus be controlled. In contrast to bulk-degrading PLAGA matrices, PHBV matrices lose very very slowly, and its adaptable properties make it a suitable matrix material for designing tissue-engineered bone.

There is a long felt need in the art for compositions and methods to enhance wound healing and organ and tissue repair. The present invention satisfies this need.

### BRIEF SUMMARY OF THE INVENTION

The present application discloses the ability of FTY720, locally released from thin biomaterial surfaces, to improve allograft vascularization, mechanical integrity, osseous remodeling, and ultimately incorporation at the host-graft interface. Specifically, devitalized bone allografts were coated with a thin polymer coating of FDA-approved 50:50 poly(lactic-co-glycolic acid) (PLAGA) encapsulated with bioactive FTY720.

The present invention provides compositions and methods useful for enhancing bone and wound healing, comprising administering a composition containing a biocompatible polymer and at least one compound having S1P receptor selective activity, or biologically active derivatives and analogs thereof. In one aspect, the activity is agonist activity. In another aspect, the activity is antagonist activity.

In one embodiment, the invention encompasses administering an effective amount of to a wound or defect in a subject in need thereof.

In one aspect, the method stimulates healing of a bone allograft.

In one aspect, the polymer of the invention is PLAGA or PHBV.

In one aspect, the composition comprising a polymer and at least one S1P receptor selective agonist or antagonist is coated on a bone allograft and the bone allograft is inserted into the bony defect. In one aspect, the agonist is FTY720, or a derivative or analog thereof.

In one aspect, PLAGA is a 50:50 or 85:15 mixture of the 72.3 kDa and 123.6 kDa forms.

In one aspect, PLAGA is mixed with methylene chloride to form a PLAGA:methylene chloride solution. In one aspect, PLAGA is mixed with methylene chloride at weight to volume ratios of 1:10, 1:12, or 1:14. In one aspect, FTY720 or a biologically active derivative or analog thereof is added to the PLAGA:methylene chloride solution. In one aspect, FTY720 or a biologically active derivative or analog thereof is added to said PLAGA:methylene chloride solution at a ratio of about 1:200 weight:weight.

In one aspect, the composition of the invention includes additional ingredients, including but not limited to additional therapeutic agents and optionally at least one purified antimicrobial agent. The composition of the invention comprising at least one polymer and at least on bioactive agent, such as FTY720, can further comprise additional therapeutic additives, alone or in combination (e.g., 2, 3, or 4 additional additives). Examples of additional additives include but are not limited to: (a) antimicrobials, (b) steroids (e.g., hydrocortisone, triamcinolone); (c) pain medications (e.g., aspirin, an NSAID, and a local anesthetic); (d) anti-inflammatory agents; (e) growth factors; (f) cytokines; (g) hormones; and (h) combinations thereof.

In one embodiment, the composition is administered to tissue using a method selected from the group consisting of directly, topically, subcutaneously, and parenterally. In one aspect, the composition is administered directly.

In one embodiment, the method enhances angiogenesis.

In one embodiment, the subject is human.

In one embodiment, the compositions and methods of the invention increase the structural integrity of a bone allograft-host bone interface and restore normal bone turnover and remodeling to a defect site.

In one embodiment, the compositions and methods of the invention are useful for treating wounds. In one aspect, the wound is a wound or injury to a bone, including from surgery. In one aspect, the method enhances bone healing.

The present invention further provides kits useful for the practice of the invention. In one embodiment, the present invention provides a kit for administering a composition of the invention for treating a wound or for enhancing bone healing. In one aspect, the kit comprises a composition comprising a biologically compatible polymer and at least one S1P receptor selective agonist or antagonist, optionally a pharmaceutically acceptable carrier, optionally at least one antimicrobial agent, optionally at least one additional therapeutic agent, an applicator, and an instructional material for the use thereof.

The present invention encompasses the use of FTY720 and biologically active derivatives and analogs thereof. For example, useful compounds of the invention include:

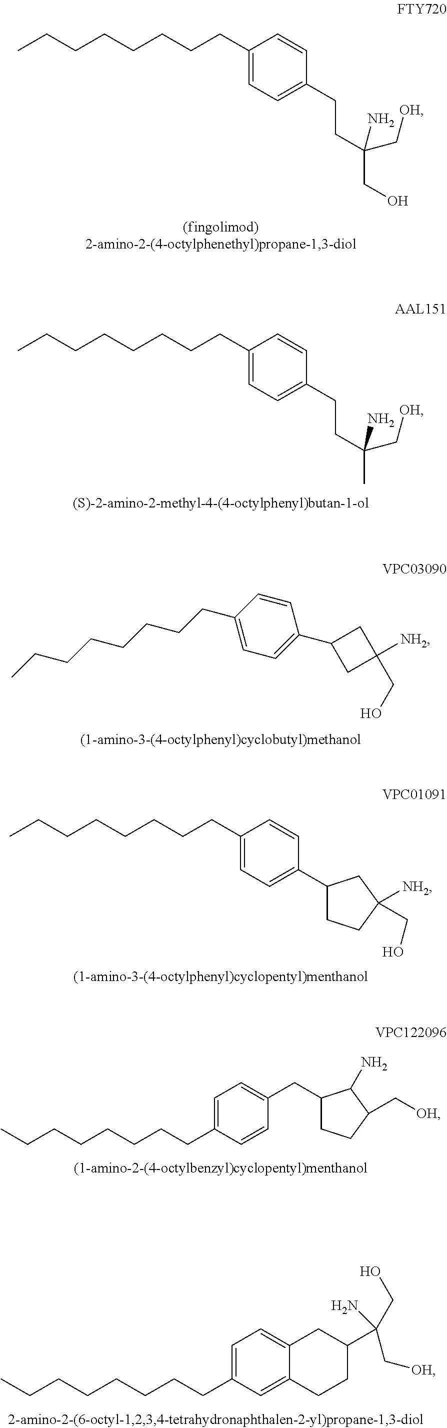

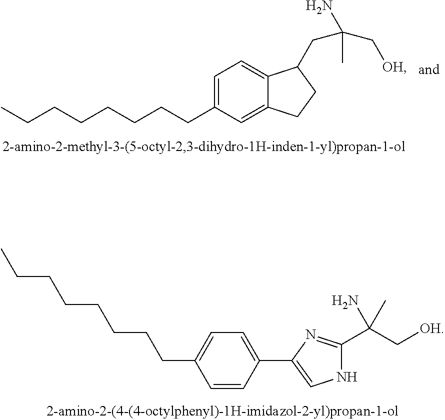

Enhancements of wound healing and bone healing or repair are described herein or are known in the art and include, but are not limited to, increases in bone density, increases in structural integrity of bone allograft-host bone interfaces, and increased deposition of bony tissue at bone allograft-host bone interfaces.

Various aspects and embodiments of the invention are described in further detail below.

### BRIEF SUMMARY OF THE DRAWINGS

FIG. 1. Characterization of PLAGA-coated allograft pore size. (A) Outline of parameters used to create various PLAGA coatings, where average pore size before and after coating and total volume of PLAGA coating is calculated for each sample using microCT evaluation. (B) Four macroscale images (left—uncoated; right—coated) of allograft cross-sections with representative polymer coating. Images show complete obstruction of smaller pores by polymer coating, whereas larger pores retain open structure. The lower panels are increased magnification images of the indicated regions of the upper panels.

FIG. 2. Characterization of PLAGA coating thickness. (A) Thickness of the PLAGA coating on the outer surface and the inner canal of the allograft, measured for each experimental group using microCT evaluation. n=4 per group. (B) Cross-sectional slice of rat femur allograft coated with PLAGA. Threshold values show bone tissue in white (200-1000) and PLAGA coating in red (112-200). Scale bar=1 mm.

FIG. 3. Characterization of polymer degradation and drug release. In vitro percent release of S1P from PLAGA-coated allografts was measured using radioactive ³³P labeling. Approximately 0.57 mg of S1P was released in 14 days with a loading efficiency of 64%. Since S1P and FTY720 have similar molecular weights and structures, we assume FTY720 exhibits similar release profiles from coated allografts.

FIG. 4. MicroCT imaging of bone remodeling. (A) Representative images show in vivo microCT low-resolution scans of segmental defects at the day of the surgery and following 6 weeks healing. Defects loaded with either uncoated allografts (U), 1:12 PLAGA coated allografts (C), or 1:12 PLAGA coated, 1:200 FTY720 loaded allografts (C/L). C/L group shows superior osseous integration particularly at the interface of the defects. Scale bar=1 mm. (B) Bone density of the host bone and allograft near the interface was calculated using microCT evaluation. The density of the C/L allograft is closest to the density of the host bone compared to the U and C groups, perhaps due to active remodeling of the bone in this group.

FIG. 5. Measurement of elastic modulus and ultimate compressive strength. (A) Results from the Instron 4511 demonstrate that the 1:12 PLAGA coated+1:200 FTY720-loaded (C/L) group had a significantly higher elastic modulus in comparison to the U and C groups. *Statistically significant compared with U and C (where p<0.05). (B) *Statistically significant compared with U and C (where p=0.081).

FIG. 6. Assessment of mural cell and leukocyte recruitment. (A) Number of blood vessels stained with smooth muscle α-actin. ^{a} Statistically significant between C/L and both U and C groups (where p<0.05). (B) Representative confocal microscopic images of SMA+ mural cells (red) within tissue sections from uncoated, (U) 1:12 PLAGA-coated (C), and 1:12 PLAGA-coated, 1:200 FTY720-loaded (C/L) allografts. (C) Representative confocal microscopic images of CD45+ leukocytes (green) within tissue sections from uncoated, (U) 1:12 PLAGA-coated (C), and 1:12 PLAGA-coated, 1:200 FTY720-loaded (C/L) allografts. Scale bar=150 mm.

FIG. 7. H&E and Masson's trichrome staining of tibial defects. (A) Uncoated (U) and (B) 1:12 PLAGA-coated (C) samples show poor allograft-host bone integration after 6 weeks healing while (C) 1:200 FTY720-loaded (C/L) group show superior osseous integration with newly-formed bony islands. Substantial osteogenesis observed in the FTY720-loaded group (C/L). Scale bar=250 mm.

### DETAILED DESCRIPTION OF THE INVENTION

### Abbreviations and Acronyms

BMP-2—bone morphogenetic protein 2
BSA—bovine serum albumin
C—coated
C/L—coated-loaded
DMEM—Dulbecco's modified Eagle's medium
ECM—extracellular matrix
ES—embryonic stem cell
FACS—fluorescent activated cell sorting
FAF—fatty acid free
FBS—fetal bovine serum
FGF—fibroblast growth factor
FTY720—fingolimod
gf—growth factor
GPCR—G-protein coupled receptor
H&E—hematoxylin and eosin
HSC—hematopoietic stem cell
HS—human serum (also referred to as HmS herein)
HSA—human serum albumin
IL-1β—interleukin-1 beta
IGF-1—insulin-like growth factor 1
MMP—matrix metalloprotease
PDGF—platelet-derived growth factor
PHBV—polyhydroxybutyrate-co-valerate
PLA—polylactide
PLAGA—poly(lactic-co-glycolic acid)
S1P—sphingosine-1-phosphate
SBF—simulated body fluid
SCGF-β—stem cell growth factor-β
SMA—smooth muscle α-actin
SMC—smooth muscle cell
TNFα—tumor necrosis factor alpha
U—unloaded
UCS—ultimate competitive strength
ULA—ultra low attachment tissue culture plate
VEGF—Vascular endothelial growth factor

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “abluminal” refers to something being directed away from the lumen of a tubular structure, i.e., a blood vessel.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease or disorder, including, but not limited to, pain and inflammation.

The term “adult” as used herein, is meant to refer to any non-embryonic or non juvenile subject. For example the term “adult adipose tissue stem cell,” refers to an adipose stem cell, other than that obtained from an embryo or juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

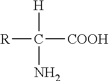

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

“Angiogenesis-associated” disease or disorder refers to a disease or disorder associated with aberrant angiogenesis or a disease or disorder reliant on angiogenesis. Changes in microvessel density are encompassed within the term “angiogenesis-associated.”

An “antagonist” is a composition of matter which when administered to a mammal such as a human, inhibits a biological activity attributable to the level or presence of a compound or molecule of interest in the subject.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes.

“Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients may be added. The term does not exclude media which have been prepared or are intended for specific uses, but which upon modification can be used for other cell types, etc.

The term “biocompatible,” as used herein, refers to a material that does not elicit a substantial detrimental response in the host.

The term “biodegradable,” as used herein, means capable of being biologically decomposed. A biodegradable material differs from a non-biodegradable material in that a biodegradable material can be biologically decomposed into units which may be either removed from the biological system and/or chemically incorporated into the biological system.

The term “biological sample,” as used herein, refers to samples obtained from a living organism, including skin, hair, tissue, blood, plasma, cells, sweat, and urine.

The term “bioresorbable,” as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable,” as the term is used herein, means that at least 80% of the total mass of material implanted is resorbed within one year.

As used herein “burn” or “burns” refer to any detectable injury to tissue caused by energy applied to the tissue. The terms “burn” or “burns” further refer to any burning, or charring of the tissue, including thermal burns caused by contact with flames, hot liquids, hot surfaces, and other sources of high heat as well as steam, chemical burns, radiation, and electrical burns. First degree burns show redness; second-degree burns show vesication; third degree burns show necrosis through the entire skin. Burns of the first and second degree are partial-thickness burns, those of the third degree are full-thickness burns.

The phrases “cell culture medium,” “culture medium” (plural “media” in each case) and “medium formulation” refer to a nutritive solution for cultivating cells and may be used interchangeably.

The term “clearance”, as used herein refers to the physiological process of removing a compound or molecule, such as by diffusion, exfoliation, removal via the bloodstream, and excretion in urine, or via sweat or other fluid.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

A “compound,” as used herein, refers to any type of substance or agent that is commonly considered a drug, or a candidate for use as a drug, combinations, and mixtures of the above, as well as polypeptides and antibodies of the invention.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

The term “decreased blood flow”, as used herein, refers to a decrease in blood flow at a site of injury, disease, or disorder, and includes, but is not limited, a decrease in flow rate, an increase in stasis, and an increase in sludging in the vessels.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect.

The terms “enhancing bone repair” or “enhancing bone healing” as used herein refer to methods of speeding up or inducing better bone repair or grafting using compounds and coatings of the invention, relative to the speed or amount of bone repair that occurs without administration of compounds and coatings of the invention. These enhancements are described herein or are known in the art and include, but are not limited to, increased allograft vascularization, increases in bone density, increases in structural integrity of bone allograft-host bone interfaces, and increased deposition of bony tissue at bone allograft-host bone interfaces. Repair or healing can be enhanced directly or indirectly.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, “feeders,” and “feeder layers” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” molecule is a molecule in a form in which it exhibits a property or activity by which it is characterized.

“Graft” refers to any free (unattached) cell, tissue, or organ for transplantation.

“Allograft” refers to a transplanted cell, tissue, or organ derived from a different animal of the same species.

“Xenograft” refers to a transplanted cell, tissue, or organ derived from an animal of a different species.

The term “growth factor” as used herein means a bioactive molecule that promotes the proliferation of a cell or tissue. Growth factors useful in the present invention include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “improved blood flow,” as used herein, refers to increased blood flow in a subject being treated according to the methods of the invention compared with the flow in a subject with an otherwise identical injury or condition not being treated according to the methods of the invention. Improved flow is determined by methods such as those described herein and can include less stasis, less sludging, or a combination of both, in the subject being treated compared with the untreated subject.

The term “ingredient” refers to any compound, whether of chemical or biological origin, that can be used in cell culture media to maintain or promote the proliferation, survival, or differentiation of cells. The terms “component,” “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block”.

“Inhibiting decreased blood flow” as described herein, refers to any method or technique which inhibits the decrease in blood flow or associated changes in blood flow following injury, or where decreased blood flow is associated with a disease or disorder, particularly thermal injury. Methods of measuring blood flow are described herein. Inhibition can be direct or indirect.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container, which contains the identified compound invention, or be shipped together with a container, which contains the identified compound. Alternatively, the instructional material may be shipped separately from the container with the intention that the instructional material and the compound be used cooperatively by the recipient.

Used interchangeably herein are the terms “isolate” and “select”.

The term “isolated”, when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays, which distinguish cell types.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, a Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to either a molecule that joins two other molecules covalently or noncovalently, e.g., through ionic or hydrogen bonds or van der Waals interactions.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “musculoskeletal” as used herein encompasses the general broad meaning of the term, i.e., an organ system that gives a subject the ability to physically move, by using the muscles and skeletal system. Apart from locomotion, the skeleton also lends support and protects internal organs. Musculoskeletal diseases include, but are not limited to, diseases of the muscles and their associated ligaments, and other connective tissue and of the bones and cartilage viewed collectively. Musculoskeletal disorders include, for example, problems such as low back pain, joint injuries and repetitive strain injuries of various sorts.

“Osteogenesis” as used herein refers to bone growth, bone remodeling, and repair of bone due to injury or disease.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The term “prevent,” as used herein, means to stop something from happening, or taking advance measures against something possible or probable from happening. In the context of medicine, “prevention” generally refers to action taken to decrease the chance of getting a disease or condition.

The term “progeny” of a stem cell as used herein refers to a cell which is derived from a stem cell and may still have all of the differentiation abilities of the parental stem cell, i.e., multipotency, or one that may no longer be multipotent, but is now committed to being able to differentiate into only one cell type, i.e., a committed cell type. The term may also refer to a differentiated cell.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

A “reversibly implantable” device is one which may be inserted (e.g. surgically or by insertion into a natural orifice of the animal) into the body of an animal and thereafter removed without great harm to the health of the animal.

As used herein, an “S1P modulating agent” refers a compound or composition that is capable of inducing a detectable change in S1P receptor activity in vivo or in vitro (e.g., at least 10% increase or decrease in S1P activity as measured by a given assay such as the bioassay described in the Examples). “S1P receptor,” as used herein, refers to all of the S1P receptor subtypes (for example, the S1P receptors S1P1, S1P2, S1P3, S1P4, and S1P5), unless the specific subtype is indicated.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, “scaffold” refers to a supporting framework, such as one for bone or tissue growth, either in vivo or in vitro.

As used herein, the term “secondary antibody” refers to an antibody that binds to the constant region of another antibody (the primary antibody).

The term “skin,” as used herein, refers to the commonly used definition of skin, e.g., the epidermis and dermis, and the cells, glands, mucosa, and connective tissue which comprise the skin.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds,” as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In one aspect, the activity or function is stimulated by at least 10% compared to a control value, more preferably by at least 25%, and even more preferably by at least 50%. The term “stimulator” as used herein, refers to any composition, compound or agent, the application of which results in the stimulation of a process or function of interest, including, but not limited to, wound healing, angiogenesis, bone healing, osteoblast production and function, and osteoclast production, differentiation, and activity.

A “subject” of diagnosis or treatment is an animal, including a human. It also includes pets and livestock.

As used herein, a “subject in need thereof” is a patient, animal, mammal, or human, who will benefit from the method of this invention.

A “surface active agent” or “surfactant” is a substance that has the ability to reduce the surface tension of materials and enable penetration into and through materials.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “thermal injury” is used interchangeably with “thermal burn” herein.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “tissue injury-associated decreased blood flow”, as used herein, refers to the decrease in blood flow which occurs following an injury, such as a wound, a fracture, a surgical procedure, or a thermal injury. The decrease in blood flow includes, but is not limited to, decreased volume, rate, stasis, or sludging. One of ordinary skill in the art will appreciate that there are multiple parameters which can be used as measures or signs of decreased blood flow, as well as multiple techniques to determine decreased blood flow.

The term “topical application,” as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous.”

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

As used herein “wound” or “wounds” may refer to any detectable break in the tissues of the body, such as injury to skin or to an injury or damage, or to a damaged site associated with a disease or disorder. As used herein, the term “wound” relates to a physical tear, break, or rupture to a tissue or cell layer. A wound may occur by any physical insult, including a surgical procedure or as a result of a disease, disorder condition. Although the terms “wound” and “injury” are not always defined exactly the same way, the use of one term herein, such as “injury”, is not meant to exclude the meaning of the other term.

### Chemical Definitions

As used herein, the term “halogen” or “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from two to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from two to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=8, represents cyclopropyl, cyclobutyl, cyclopentyl, cyclohexyl, cycloheptyl, and cyclooctyl.

As used herein the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like. “Optionally substituted aryl” includes aryl compounds having from zero to four substituents, and “substituted aryl” includes aryl compounds having one or more substituents. The term (C₅-C₈ alkyl)aryl refers to any aryl group which is attached to the parent moiety via the alkyl group.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The term “heterocyclic group” refers to an optionally substituted mono- or bicyclic carbocyclic ring system containing from one to three heteroatoms wherein the heteroatoms are selected from the group consisting of oxygen, sulfur, and nitrogen.

As used herein the term “heteroaryl” refers to an optionally substituted mono- or bicyclic carbocyclic ring system having one or two aromatic rings containing from one to three heteroatoms and includes, but is not limited to, furyl, thienyl, pyridyl and the like.

As used herein, the term “optionally substituted” refers to from zero to four substituents, wherein the substituents are each independently selected. Each of the independently selected substituents may be the same or different than other substituents.

The compounds of the present invention contain one or more asymmetric centers in the molecule. In accordance with the present invention a structure that does not designate the stereochemistry is to be understood as embracing all the various optical isomers, as well as racemic mixtures thereof.

The compounds of the present invention may exist in tautomeric forms and the invention includes both mixtures and separate individual tautomers. For example the following structure:

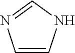

is understood to represent a mixture of the structures:

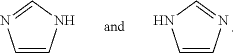

The terminology used herein is for the purpose of describing the particular versions or embodiments only, and is not intended to limit the scope of the present invention. All publications mentioned herein are incorporated by reference in their entirety.

### Embodiments

Existing wound healing formulations can also be used as pharmaceutically acceptable carriers for the procedures described herein.

Some examples of wounds, defects, diseases, and disorders that may be treated according to the methods of the invention are discussed herein. The invention should not be construed as being limited solely to these examples, as other wounds, defects, diseases, and disorders that are not described herein, or at present unknown, once known, may also be treatable using the methods of the invention.

The present invention provides for the use of bioactive polymer compositions for the compositions and methods of the invention, including, but not limited to, the polymers PLAGA and PHBV and bioactive molecules including, but not limited to FTY720 and S1P, and biologically active analogs and derivatives thereof. These polymers are biocompatible and biodegradable.

The present invention can also be practiced with other effective polymers, and one of ordinary skill in the art will appreciate how to choose and use those suitable effective polymers.

The polymer composition comprising at least one bioactive agent, including, but not limited to the bioactive agent FTY720 and active derivatives and analogs thereof, can be applied to materials other than bone graft material as exemplified herein. The structures of S1P and the S1P receptor agonist FTY720 are provided below.

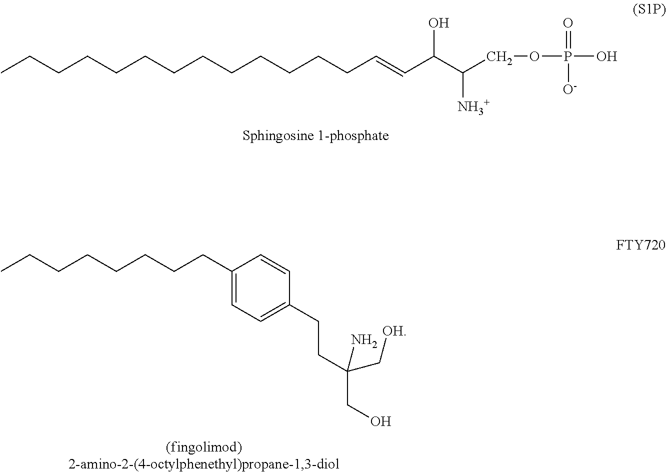

In one aspect, FTY720, or biologically active derivatives and analogs thereof are phosphorylated. In one aspect, other S1P receptor agonists which stimulate the same activity as FTY720 are used. In one aspect, the compound is in the form of a salt or an ester.

Support for other known compounds that are biologically active analogs and derivatives of S1P and FTY720 and their synthesis is available in the art and can be found, for example, in U.S. Pat. Nos. 7,241,790, 7,560,477, and 7,638,637, in U.S. patent application Ser. Nos. 12/179,816, 12/470,011, 12/470,017, 12/189,010, and 12/470,009, and in PCT Pat. App. WO US/2009/023854.

Injuries, Wounds, Diseases, and Disorders

A subject having a site of injury or wound, or in some cases a disease or disorder, may be susceptible to decreased blood flow at that site and therefore be in need of treatment. In one aspect, the decreased blood flow is in microvessels. These conditions may typically arise from many types of injury including trauma, surgery, and trauma to the skin and/or exposed soft tissue, resulting in an inflammatory reaction and decreased blood flow, particularly in the microvasculature. The types of injuries, disease, and disorders encompassed by the methods of the invention therefore include, bone trauma, diseases, and disorders, burns, chronic wounds, and surgical procedures such as microvascular surgery, skin flaps and skin grafts, and tissue injury resulting from, for example, a burn, scrape, cut, incision, laceration, ulcer, body piercing, bite wound, trauma, stab wound, gunshot wound, surgical wound, stretch injury, crush wound, compression wound, fracture, sprain, strain, stroke, infarction, aneurysm, herniation, ischemia, fistula, dislocation, radiation, cell, tissue or organ grafting and transplantation, injuries sustained during medical procedures, or cancer.

Such injuries include, but are not limited to, bone injury, skin injury, muscle injury, brain injury, eye injury, or spinal cord injury. Tissue injury can include joint injury, back injury, heart injury, vascular system injury, soft tissue injury, cartilage injury, lymphatic system injury, tendon injury, ligament injury, or abdominal injury.

While it is important to treat any condition in which the potential for cell or tissue damage exists immediately (e.g., an acute wound), it is essential that certain conditions be treated before they become chronic conditions. Chronic diseases are a challenge to the patient, the health care professional, and to the health care system. They significantly impair the quality of life for millions of people in the United States. Intensive treatment is required with a high cost to society in terms of lost productivity and health care dollars. The management of chronic diseases can place an enormous strain on health care resources. Diseases or conditions, for example, wounds that were once acute but have progressed to chronic often do so because the diseases cannot be controlled or treated with known therapies. Therefore, there is a need for improved therapies for treating chronic diseases and conditions characterized by cell and tissue damage.

Other non-limiting examples of wounds suitable for treatment in accordance with the present disclosure include trauma, fractures, animal bites, arterial disease, insect stings and bites, bone infections, compromised skin/muscle grafts, gangrene, skin tears or lacerations, surgical incisions, including slow or non-healing surgical wounds, and post-operation infections. It is understood, that the listed wounds are non-limiting and that only a portion of wounds suitable for treatment in accordance with the present disclosure are listed herein.

Additional Therapeutic Agents and Ingredients

The composition of the invention comprising at least one polymer and at least on bioactive agent, such as FTY720, can further comprise additional therapeutic additives, alone or in combination (e.g., 2, 3, or 4 additional additives). Examples of additional additives include but are not limited to: (a) antimicrobials, (b) steroids (e.g., hydrocortisone, triamcinolone); (c) pain medications (e.g., aspirin, an NSAID, and a local anesthetic); (d) anti-inflammatory agents; (e) growth factors; (f) cytokines; (g) hormones; and (h) combinations thereof.

The types of drugs and specific drugs within categories which are encompassed within the invention are intended to be non-limiting examples.

In one embodiment, a formulation of the invention contains an antimicrobial agent. The antimicrobial agent may be provided at, for example, a standard therapeutically effective amount. A standard therapeutically effective amount is an amount that is typically used by one of ordinary skill in the art or an amount approved by a regulatory agency (e.g., the FDA or its European counterpart). Antimicrobial agents useful for the invention include those directed against the spectra of gram positive organisms, gram negative organisms, fungi, and viruses.

The present invention provides for the use of anesthetics. According to the topical anesthetic embodiment of the present invention, in one aspect, suitable local anesthetic agents having a melting point of 30° to 70° C. are prilocalne, tetracaine, butanilcaine, trimecaine, benzocaine, lidocaine, bupivocaine, dibucaine, mepivocaine, and etidocaine.

The present invention further encompasses the use of at least two anesthetics.

The local anesthetic composition of the present invention may further comprise suitable additives, such a pigment, a dye, an anti-oxidant, a stabilizer or a fragrance provided that addition of such an additive does not destroy the single phase of the anesthetic composition.

In one aspect, the hydrated local anesthetic mixture is prepared by melting the local anesthetic with the higher melting point of the two, followed by addition of the other local anesthetic, under vigorous mechanical mixing, such as trituration or grinding. A milky viscous liquid is formed, at which point, the surfactant is added with more mechanical mixing. Mixing of the surfactant produces a milky liquid of somewhat lower viscosity. Finally, the balance of water is added under vigorous mechanical mixing. The material can then be transferred to an air tight container, after which a clear composition is obtained after about 60 minutes at room temperature.

Alternatively, the hydrated local anesthetic mixture can be prepared by first melting the lower melting local anesthetic, followed by addition of the other local anesthetic along with vigorous mechanical mixing, then addition of the surfactant and water as above. However, when the lower melting local anesthetic is melted first, the storage time needed to obtain the single-phase composition, increases from about 1 hour to about 72 hours. Accordingly, the former method is preferred.

One of ordinary skill in the art will appreciate that there are multiple suitable surfactants useful for preparing the hydrated topical anesthetic of the present invention. For example, single-phase hydrated topical anesthetics can be prepared from anionic, cationic, or non-ionic surfactants.

Several embodiments include use of any therapeutic molecule including, without limitation, any pharmaceutical or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfuram and disulfuram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All substances listed by the U.S. Pharmacopeia are also included within the substances of the present invention.

Antimicrobial agents include, but are not limited to: silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), Neosporin (i.e., Bacitracin, Polymyxin B, and Neomycin), Polysporin (i.e., Bacitracin and Polymyxin B). Additional antimicrobials include topical antimicrobials (i.e., antiseptics), examples of which include silver salts, iodine, benzalkonium chloride, alcohol, hydrogen peroxide, and chlorhexidine.

Analgesic: Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin.

Antihypertensive: Aflyzosin Hydrochloride; Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Dilevalol Hydrochloride; Diltiazem Malate; Ditekiren; Doxazosin Mesylate; Ecadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone; Indapamide; Indolaprif Hydrochloride; Indoramin; Indoramin Hydrochloride; Indorenate Hydrochloride; Lacidipine; Leniquinsin; Levcromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosine; Minoxidil; Monatepil Maleate; Muzolimine; Nebivolol; Nitrendipine; Ofornine; Pargyline Hydrochloride; Pazoxide; Pelanserin Hydrochloride; Perindopril Erbumine; Phenoxybenzamine Hydrochloride; Pinacidil; Pivopril; Polythiazide; Prazosin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sulfinalol Hydrochloride; Tasosartan; Teludipine Hydrochloride; Temocapril Hydrochloride; Terazosin Hydrochloride; Terlakiren; Tiamenidine; Tiamenidine Hydrochloride; Ticrynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine.

Anti-inflammatory: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Growth Factors

In one embodiment, an effective amount of at least one growth factor, cytokine, hormone, or extracellular matrix compound or protein useful for enhancing wound healing is administered as part of the composition. In another aspect, one or more growth factors are administered separately from the polymer:S1P receptor agonist composition. In one aspect, a combination of these agents is used. In one aspect, growth factors useful in the practice of the invention include, but are not limited to, EGF, PDGF, GCSF, IL6, IL8, IL10, MCP1, MCP2, Tissue Factor, FGFb, KGF, VEGF, PDGF, MMP1, MMP9, TIMP1, TIMP2, TGFβ, interferons, and HGF. One of ordinary skill in the art will appreciate that the choice of growth factor, cytokine, hormone, or extracellular matrix protein used will vary depending on criteria such as the type of injury, disease, or disorder being treated, the age, health, sex, and weight of the subject, etc. In one aspect, the growth factors, cytokines, hormones, and extracellular matrix compounds and proteins are human.

Proteins and other biologically active compounds that can be incorporated into, or included as an additive within, a composition comprising compounds of the present invention include, but are not limited to, collagen (including cross-linked collagen), fibronectin, laminin, elastin (including cross-linked elastin), osteopontin, osteonectin, bone sialoproteins (Bsp), alpha-2HS-glycoproteins, bone Gla-protein (Bgp), matrix Gla-protein, bone phosphoglycoprotein, bone phosphoprotein, bone proteoglycan, protolipids, bone morphogenetic protein, cartilage induction factor, skeletal growth factor, enzymes, or combinations and biologically active fragments thereof. Adjuvants that diminish an immune response can also be used in conjunction with the composite of the subject invention.

Other molecules useful as compounds or substances in the present invention include, but are not limited to, growth hormones, leptin, leukemia inhibitory factor (LIF), tumor necrosis factor alpha and beta, endostatin, angiostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18. Embodiments involving amino acids, peptides, polypeptides, and proteins may include any type of such molecules of any size and complexity as well as combinations of such molecules.

Pharmaceutical Compositions and Delivery Form

The formulations of the invention may be prepared in a variety of forms known in the art, such as liquids, aerosols, or gels, if not used in a polymer composition, or the active ingredient can be added to the polymer solution. Topical administration of the present formulation can be performed by, for example, hand, mechanically (e.g., extrusion and spray delivery) or as a component of a dressing (e.g., gauze or other wound covering). The administration of the formulation directly by hand or as described herein to a tissue or surface, such as an allograft, is preformed to achieve a therapeutic coating, which may be uniform, alone or in combination with an overlying dressing.

Delivery of the bioactive ingredients is not limited to the polymers described herein, but also includes, but is not limited to, hydrogels, PEG, polysaccharides, alginate, chitosan, and lipid coatings.

In one embodiment, the administration of the formulation mechanically is performed by using a device that physically pushes the composition onto a tissue or biomaterial surface to achieve a therapeutic coating, which may be uniform, alone or in combination with an overlying dressing. In one aspect, the material, such as an allograft, is bathed in the solution.

In another embodiment, the formulation can be sprayed onto a tissue or biomaterial surface to achieve a therapeutic coating, which may be uniform, alone or in combination with an overlying dressing. When part of a dressing, the formulation is applied to achieve a therapeutic coating of the surface, which may be uniform.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 70% (w/w) active ingredient, although the concentration of the active ingredient may be as high as the solubility limit of the active ingredient in the solvent. Formulations for topical administration may further comprise one or more of the additional ingredients described herein.

Those of ordinary skill in the art will be able to identify readily those pharmaceutical agents that have utility with the present invention. Those of ordinary skill in the art will also recognize numerous other compounds that fall within the categories and that are useful according to the invention for treating injuries where reduced blood flow occurs.

The invention encompasses the preparation and use of compositions useful for treatment of various skin related injuries, trauma, diseases, disorders, or conditions described herein, including burns, wounds, surgical incisions, etc. The invention also encompasses other injuries, trauma, associated diseases, and disorders other than those of the skin, including, but not limited to, gum diseases and disorders. Such a composition may consist of the polymer and the active ingredient alone, in a form suitable for administration to a subject or the composition may comprise at least one active ingredient and one or more pharmaceutically acceptable carriers, one or more additional ingredients, or some combination of these. The active ingredient may be present in the composition in the form of a physiologically acceptable ester or salt, such as in combination with a physiologically acceptable cation or anion, as is well known in the art.

An obstacle for topical administration of pharmaceuticals to the skin is the stratum corneum layer of the epidermis. The stratum corneum is a highly resistant layer comprised of protein, cholesterol, sphingolipids, free fatty acids and various other lipids, and includes cornified and living cells. One of the factors that limits the penetration rate (flux) of a compound through the stratum corneum is the amount of the active substance, which can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The compounds of the invention may be administered to, for example, a cell, a tissue, or a subject by any of several methods described herein and by others which are known to those of skill in the art.

The relative amounts of the active ingredient, the pharmaceutically acceptable carrier, and any additional ingredients in a pharmaceutical composition of the invention will vary, depending upon the identity, sex, age, size, and condition of the subject treated and further depending upon the route by which the composition is to be administered.

In addition to the active ingredient, a composition of the invention may further comprise one or more additional pharmaceutically active or therapeutic agents. Particularly contemplated additional agents include anti-emetics and scavengers such as cyanide and cyanate scavengers.

Controlled- or sustained-release formulations of a composition of the invention may be made using conventional technology.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Additionally, formulations for topical administration may include liquids, ointments, lotions, creams, gels (e.g., poloxamer gel), drops, suppositories, sprays, aerosols, and powders. Conventional pharmaceutical carriers, aqueous, powder or oily bases, thickeners and the like may be necessary or desirable. The disclosed compositions can be administered, for example, in a microfiber, polymer (e.g., collagen), nanosphere, aerosol, lotion, cream, fabric, plastic, tissue engineered scaffold, matrix material, tablet, implanted container, powder, oil, resin, wound dressing, bead, microbead, slow release bead, capsule, injectables, intravenous drips, pump device, silicone implants, or any bio-engineered materials. Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

The source of active compound to be formulated will generally depend upon the particular form of the compound. Small organic molecules and peptidyl or oligo fragments can be chemically synthesized and provided in a pure form suitable for pharmaceutical/cosmetic usage. Products of natural extracts can be purified according to techniques known in the art. Recombinant sources of compounds are also available to those of ordinary skill in the art.

In alternative embodiments, the topically active pharmaceutical composition may be optionally combined with other ingredients such as moisturizers, cosmetic adjuvants, anti-oxidants, chelating agents, bleaching agents, tyrosinase inhibitors, and other known depigmentation agents, surfactants, foaming agents, conditioners, humectants, wetting agents, emulsifying agents, fragrances, viscosifiers, buffering agents, preservatives, sunscreens, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art. The compositions of this invention may also contain active amounts of retinoids (i.e., compounds that bind to any members of the family of retinoid receptors), including, for example, tretinoin, retinol, esters of tretinoin and/or retinol and the like.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

The present invention encompasses biologically active analogs, homologs, derivatives, and modifications of the compounds of the invention. Methods for the preparation of such compounds are known in the art.

Liquid derivatives and natural extracts made directly from biological sources may be employed in the compositions of this invention in a concentration (w/w) from about 1 to about 99%. Fractions of natural extracts and protease inhibitors may have a different preferred rage, from about 0.01% to about 20% and, more preferably, from about 1% to about 10% of the composition. Of course, mixtures of the active agents of this invention may be combined and used together in the same formulation, or in serial applications of different formulations.

The composition of the invention may comprise a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition of the invention such as a therapeutic gel or cream. Examples of preservatives useful in accordance with the invention included but are not limited to those selected from the group consisting of benzyl alcohol, sorbic acid, parabens, imidurea, and combinations thereof A particularly preferred preservative is a combination of about 0.5% to 2.0% benzyl alcohol and 0.05% to 0.5% sorbic acid.

The composition may include an antioxidant and a chelating agent which inhibit the degradation of the compound for use in the invention in the aqueous gel formulation. Preferred antioxidants for some compounds are BHT, BHA, alphatocopherol, and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are preferred antioxidant and chelating agent respectively for some compounds, other suitable and equivalent antioxidants and chelating agents may be substituted therefor as would be known to those skilled in the art.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Other components such as preservatives, antioxidants, surfactants, absorption enhancers, viscosity enhancers or film forming polymers, bulking agents, diluents, coloring agents, flavoring agents, pH modifiers, sweeteners or taste-masking agents may also be incorporated into the composition. Suitable coloring agents include red, black, and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

Absorption enhancers for use in accordance with the present invention include, for example, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin. In certain preferred embodiments wherein an absorption enhancer is included in the formulation, the absorption enhancer is included in an amount of from about 0.001% to about 10% by weight of the formulation, preferably in an amount of about 0.01% to about 5% by weight of the formulation.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, and birds including commercially relevant birds such as chickens, ducks, geese, and turkeys.

The pharmaceutical compositions of the invention can be administered in any suitable formulation, by any suitable means, and by any suitable route of administration. Formulations suitable for topical administration include, but are not limited to, liquid or semi-liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Topical administration of compositions of the invention may include transdermal application. Transdermal application can be performed either passively or using iontophoresis or electroporation.

Compositions of the invention may be applied using transdermal patches. Transdermal patches are adhesive backed patches laced with an effective amount of compounds of the invention. The pressure-sensitive adhesive of the matrix will normally be a solution of polyacrylate, a silicone, or polyisobutylene (PIB). Such adhesives are well known in the transdermal art. See, for instance, the Handbook of Pressure Sensitive Adhesive Technology, 2nd Edition (1989) Van Nostrand, Reinhold.

Pressure sensitive solution polyacrylate adhesives for transdermal patches are made by copolymerizing one or more acrylate monomers (“acrylate” is intended to include both acrylates and methacrylates), one or more modifying monomers, and one or more functional group-containing monomers in an organic solvent. The acrylate monomers used to make these polymers are normally alkyl acrylates of 4-17 carbon atoms, with 2-ethylhexyl acrylate, butyl acrylate, and isooctyl acrylate being preferred. Modifying monomers are typically included to alter the Tg of the polymer. Such monomers as vinyl acetate, ethyl acrylate and methacrylate, and methyl methacrylate are useful for this purpose. The functional group-containing monomer provides sites for crosslinking. The functional groups of these monomers are preferably carboxyl, hydroxy or combinations thereof. Examples of monomers that provide such groups are acrylic acid, methacrylic acid and hydroxy-containing monomers such as hydroxyethyl acrylate. The polyacrylate adhesives are preferably crosslinked using a crosslinking agent to improve their physical properties, (e.g., creep and shear resistance). The crosslinking density should be low since high degrees of crosslinking may affect the adhesive properties of the copolymer adversely. Examples of crosslinking agents are disclosed in U.S. Pat. No. 5,393,529. Solution polyacrylate pressure sensitive adhesives are commercially available under tradenames such as GELVA™ and DURO-TAK™ from 3M.

Polyisobutylene adhesives are mixtures of high molecular weight (HMW) PIB and low molecular weight (LMW) PIB. Such mixtures are described in the art, e.g., PCT/US91/02516. The molecular weight of the HMW PIB will usually be in the range of about 700,000 to 2,000,000 Da, whereas that of the LMW PIB will typically range between 35,000 to 60,000. The molecular weights referred to herein are weight average molecular weight. The weight ratio of HMW PIB to LMW PIB in the adhesive will normally range between 1:1 to 1:10. The PIB adhesive will also normally include a tackifier such as polybutene oil and high Tg, low molecular weight aliphatic resins such as the ESCOREZ™ resins available from Exxon Chemical. Polyisobutylene polymers are available commercially under the tradename VISTANEX™ from Exxon Chemical.

The silicone adhesives that may be used in forming the matrix are typically high molecular weight polydimethyl siloxanes or polydimethyldiphenyl siloxanes. Formulations of silicone adhesives that are useful in transdermal patches are described in U.S. Pat. Nos. 5,232,702, 4,906,169, and 4,951,622.

The present invention provides a system for the direct application of compounds of the invention, including additional therapeutic agents such as anesthetic agents, by iontophoresis for the treatment of decreased blood flow and concurrent pain associated with injuries, diseases, and disorders. While many compounds may be useful with the invention, as will be discussed below, it is particularly useful for the delivery of anesthetic agents such as lidocaine, bupivicaine, ropivicaine, and mepivicaine to damaged skin.

In one embodiment, the methods of the invention provide a patch device with a donor or delivery chamber that is designed to be applied directly over an injury, incision, or wound site and utilizes an electric field to stimulate delivery of the active compound or additional therapeutic agents(s). The patch is sterilized so that risk of infection is minimal. Additionally, the system delivers medication in a constant manner over an extended period of time. Generally, such time periods are at least 30 minutes and may extend to as many as 96 hours.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute about 50% to about 99.9% (w/w) of the composition, and the active ingredient may constitute about 0.1% to about 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to about 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as about 0.1% (w/w) and as much as about 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, comprise about 0.1% to about 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or atomized formulations, when dispersed, preferably have an average particle or droplet size in the range from about 0.1 to about 200 nanometers, and may further comprise one or more of the additional ingredients described herein. Additionally, the formulation taken orally can be prepared as a pharmaceutical composition, including, but not limited to, a paste, a gel, a toothpaste, a mouthwash, a solution, an oral rinse, a suspension, an ointment, a cream, and a coating.

A pharmaceutical composition of the invention may be prepared, packaged, or sold in a formulation suitable for ophthalmic administration. Such formulations may, for example, be in the form of eye drops including, for example, a 0.1% to 1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

A pharmaceutical composition of the invention may be prepared, packaged, or sold in a formulation suitable for intramucosal administration. The present invention provides for intramucosal administration of compounds to allow passage or absorption of the compounds across mucosa. Such type of administration is useful for absorption orally (gingival, sublingual, buccal, etc.), rectally, vaginally, pulmonary, nasally, etc.

In some aspects, sublingual administration has an advantage for active ingredients, as well as additional therapeutic agents, which in some cases, when given orally, are subject to a substantial first pass metabolism and enzymatic degradation through the liver, resulting in rapid metabolization and a loss of therapeutic activity related to the activity of the liver enzymes that convert the molecule into inactive metabolites, or the activity of which is decreased because of this bioconversion.

In some cases, a sublingual route of administration is capable of producing a rapid onset of action due to the considerable permeability and vascularization of the buccal mucosa. Moreover, sublingual administration can also allow the administration of active ingredients which are not normally absorbed at the level of the stomach mucosa or digestive mucosa after oral administration, or alternatively which are partially or completely degraded in acidic medium after ingestion of, for example, a tablet.

The compounds of the invention can be prepared in a formulation or pharmaceutical composition appropriate for administration that allows or enhances absorption across mucosa. Mucosal absorption enhancers include, but are not limited to, a bile salt, fatty acid, surfactant, or alcohol. In specific embodiments, the permeation enhancer can be sodium cholate, sodium dodecyl sulphate, sodium deoxycholate, taurodeoxycholate, sodium glycocholate, dimethylsulfoxide, or ethanol. In a further embodiment, a compound of the invention can be formulated with a mucosal penetration enhancer to facilitate delivery of the compound. The formulation can also be prepared with pH optimized for solubility, drug stability, and absorption through mucosa such as nasal mucosa, oral mucosa, vaginal mucosa, respiratory, and intestinal mucosa.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compounds include polyol compounds, such as oligo-, di- and monosaccharides such as sucrose, mannitol, lactose, L-arabinose, D-erythrose, D-ribose, D-xylose, D-mannose, D-galactose, lactulose, cellobiose, gentibiose, glycerin, and polyethylene glycol. Other examples of hydrophilic low molecular weight compounds useful as carriers within the invention include N-methylpyrrolidone, and alcohols (e.g., oligovinyl alcohol, ethanol, ethylene glycol, propylene glycol, etc.). These hydrophilic low molecular weight compounds can be used alone or in combination with one another or with other active or inactive components of the intranasal formulation.

When a controlled-release pharmaceutical preparation of the present invention further contains a hydrophilic base, many options are available for inclusion. Hydrophilic polymers such as a polyethylene glycol and polyvinyl pyrrolidone, sugar alcohols such as D-sorbitol and xylitol, saccharides such as sucrose, maltose, lactulose, D-fructose, dextran, and glucose, surfactants such as polyoxyethylene-hydrogenated castor oil, polyoxyethylene polyoxypropylene glycol, and polyoxyethylene sorbitan higher fatty acid esters, salts such as sodium chloride and magnesium chloride, organic acids such as citric acid and tartaric acid, amino acids such as glycine, beta-alanine, and lysine hydrochloride, and aminosaccharides such as meglumine are given as examples of the hydrophilic base. Polyethylene glycol, sucrose, and polyvinyl pyrrolidone are preferred and polyethylene glycol are further preferred. One or a combination of two or more hydrophilic bases can be used in the present invention.

The present invention contemplates pulmonary, nasal, or oral administration through an inhaler. In one embodiment, delivery from an inhaler can be a metered dose.

An inhaler is a device for patient self-administration of at least one compound of the invention comprising a spray inhaler (e.g., a nasal, oral, or pulmonary spray inhaler) containing an aerosol spray formulation of at least one compound of the invention and a pharmaceutically acceptable dispersant. In one aspect, the device is metered to disperse an amount of the aerosol formulation by forming a spray that contains a dose of at least one compound of the invention effective to treat a disease or disorder encompassed by the invention. The dispersant may be a surfactant, such as, but not limited to, polyoxyethylene fatty acid esters, polyoxyethylene fatty acid alcohols, and polyoxyethylene sorbitan fatty acid esters. Phospholipid-based surfactants also may be used.

In other embodiments, the aerosol formulation is provided as a dry powder aerosol formulation in which a compound of the invention is present as a finely divided powder. The dry powder formulation can further comprise a bulking agent, such as, but not limited to, lactose, sorbitol, sucrose, and mannitol.

In another specific embodiment, the aerosol formulation is a liquid aerosol formulation further comprising a pharmaceutically acceptable diluent, such as, but not limited to, sterile water, saline, buffered saline and dextrose solution.

In further embodiments, the aerosol formulation further comprises at least one additional compound of the invention in a concentration such that the metered amount of the aerosol formulation dispersed by the device contains a dose of the additional compound in a metered amount that is effective to ameliorate the symptoms of disease or disorder disclosed herein when used in combination with at least a first or second compound of the invention.

Compounds of the invention will be prepared in a formulation or pharmaceutical composition appropriate for nasal administration. In a further embodiment, the compounds of the invention can be formulated with a mucosal penetration enhancer to facilitate delivery of the drug. The formulation can also be prepared with pH optimized for solubility, drug stability, absorption through nasal mucosa, and other considerations.

Capsules, blisters, and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

For administration by inhalation, the compounds for use according to the methods of the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of e.g., gelatin for use in an inhaler or insufflator may be formulated containing a powder mix of the drugs and a suitable powder base such as lactose or starch.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compounds of the invention which may be administered to an animal, preferably a human, range in amount from about 1.0 μg to about 100 g per kilogram of body weight of the animal. The precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compounds may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The composites of the bioactive coating or it constituents of the present invention can be used as a vehicle for the in situ delivery of biologically active agents. The biologically active agents incorporated into, or included as an additive within, the composite of the subject invention can include, without limitation, medicaments, growth factors, vitamins, mineral supplements, substances used for the treatment, prevention, diagnosis, cure or mitigation of disease or illness, substances which affect the structure or function of the body, or drugs. The biologically active agents can be used, for example, to facilitate implantation of the composite or cell suspension into a subject to promote subsequent integration and healing processes. The active agents include, but are not limited to, antifungal agents, antibacterial agents, anti-viral agents, anti-parasitic agents, growth factors, angiogenic factors, anesthetics, mucopolysaccharides, metals, cells, and other wound healing agents. Because the processing conditions can be relatively benign (physiological temperature and pH), live cells can be incorporated into the composite during its formation, or subsequently allowed to infiltrate the composite through tissue engineering techniques.

Compositions comprising the compounds and bioactive coatings of the invention can be employed in any suitable manner to facilitate the growth and differentiation of the desired tissue. In other embodiments, the structure is implanted within the host animal directly at the site in which it is desired to grow the tissue or structure. In still another embodiment, the composition can be engrafted onto a host, where it will grow and mature until ready for use. Thereafter, the mature structure (or anlage) is excised from the host and implanted into the host, as appropriate.

Methods for measuring bone and wound healing are known in the art and include various cellular, molecular, biochemical, and histological techniques.

In accordance with one embodiment of the invention, compositions comprising cells and compounds of the invention are used to enhance bone and wound healing, and/or treat patients having deficient bone and wound healing.

Existing bone and wound healing formulations can also be used as pharmaceutically acceptable carriers for the procedures described herein.

The compositions and bioactive coatings and ingredients of the present invention may be administered to a subject alone or in admixture with a composition useful in the repair of bones and wounds and other defects. Such compositions include, but are not limited to bone morphogenetic proteins, hydroxyapatite/tricalcium phosphate particles (HA/TCP), gelatin, poly-L-lysine, and collagen.

Injuries, wounds and defects to which the present inventive method is useful in promoting healing, but are not limited to, broken or defective bones, abrasions, avulsions, blowing wounds, burn wounds, contusions, gunshot wounds, incised wounds, open wounds, penetrating wounds, perforating wounds, puncture wounds, seton wounds, stab wounds, surgical wounds, subcutaneous wounds, diabetic lesions, or tangential wounds. The method need not achieve complete healing of the wound or defect; it is sufficient for the method to promote any degree of wound healing or correction of the defect. In this respect, the method can be employed alone or as an adjunct to other methods for healing wounded tissue.

In one embodiment, the compositions, bioactive agents and coatings and methods of the invention are useful for disease therapy, tissue repair, transplantation, and treatment of organ, tissue, or cellular debilitation.

The compositions of the present invention can be used as a vehicle for the in situ delivery of biologically active agents. The biologically active agents incorporated into, or included as an additive within, the composite of the subject invention can include, without limitation, medicaments, growth factors, vitamins, mineral supplements, substances used for the treatment, prevention, diagnosis, cure or mitigation of disease or illness, substances which affect the structure or function of the body, or drugs. The biologically active agents can be used, for example, to facilitate implantation of the composite or cell suspension into a subject to promote subsequent integration and healing processes. The active agents include, but are not limited to, antifungal agents, antibacterial agents, anti-viral agents, anti-parasitic agents, growth factors, angiogenic factors, anesthetics, mucopolysaccharides, metals, cells, and other wound healing agents. Because the processing conditions can be relatively benign (physiological temperature and pH), live cells can be incorporated into the composite during its formation, or subsequently allowed to infiltrate the composite through tissue engineering techniques.

Non-synthetic matrix proteins like collagen, glycosaminoglycans, and hyaluronic acid, which are enzymatically digested in the body, are useful for delivery (see U.S. Pat. Nos. 4,394,320; 4,472,840; 5,366,509; 5,606,019; 5,645,591; and 5,683,459) and are suitable for use with the present invention. Other implantable media and devices can be used for delivery of the compounds and bioactive coatings of the invention in vivo. These include, but are not limited to, sponges, such as those from Integra, fibrin gels, scaffolds formed from sintered microspheres of polylactic acid glycolic acid copolymers (PLAGA), and nanofibers formed from native collagen, as well as other proteins. The compounds of the present invention can be further combined with growth factors, nutrient factors, pharmaceuticals, calcium-containing compounds, anti-inflammatory agents, antimicrobial agents, or any other substance capable of expediting or facilitating bone or tissue growth, stability, and remodeling.

The compositions of the present invention can also be combined with inorganic fillers or particles. For example for use in implantable grafts the inorganic fillers or particles can be selected from hydroxyapatite, tri-calcium phosphate, ceramic glass, amorphous calcium phosphate, porous ceramic particles or powders, mesh titanium or titanium alloy, or particulate titanium or titanium alloy.

In one embodiment, a composition of the invention is administered locally by injection. Compositions may further comprise cells. Compositions can be further combined with known drugs, and in one embodiment, the drugs are bound to the bioactive coating material. These compositions can also be prepared in the form of an implantable device that can be molded to a desired shape. In one embodiment, a graft construct is prepared comprising a biocompatible matrix and one or more cells of the present invention, wherein the matrix is formed in a shape to fill a gap or space created by the removal of a tumor, injured, or diseased tissue.

Compositions comprising bioactive coatings or materials of the invention can be employed in any suitable manner to facilitate the healing, growth, and differentiation of the desired tissue. For example, the composition can be constructed using three-dimensional or stereotactic modeling techniques. To direct the growth and differentiation of the desired structure, the composition can be cultured ex vivo in a bioreactor or incubator, as appropriate. In other embodiments, the structure is implanted within the host animal directly at the site in which it is desired to grow the tissue or structure. In still another embodiment, the composition can be engrafted onto a host, where it will grow and mature until ready for use. Thereafter, the mature structure (or anlage) is excised from the host and implanted into the host, as appropriate.

Matrices suitable for inclusion into the composition can be derived from various sources. As discussed above, the cells, matrices, and compositions of the invention can be used in tissue engineering and regeneration. Thus, the invention pertains to an implantable structure (i.e., an implant) incorporating any of these inventive features. The exact nature of the implant will vary according to the intended use. The implant can be, or comprise, as described, mature or immature tissue. Thus, for example, one type of implant can be a bone implant, comprising a population of the inventive cells that are undergoing (or are primed for) osteoblastic, adipose, chondrogenic, or osteoclastic differentiation, optionally seeded within a matrix material. Such an implant can be applied or engrafted to encourage the generation or regeneration of mature bone or other tissue within the subject.

One of ordinary skill in the art would appreciate that there are other carriers useful for delivering the compositions and compounds of the invention. Such carriers include, but are not limited to, calcium phosphate, hydroxyapatite, and synthetic or natural polymers such as collagen or collagen fragments in soluble or aggregated forms. In one aspect, such carriers serve to deliver the compositions, coatings, as well as organ, tissue, or cells to a location or to several locations. In another aspect, the compositions and compounds can be delivered either through systemic administration or by implantation. Implantation can be into one site or into several sites.

As indicated above, cells can be seeded onto and/or within the organic/inorganic composites of the present invention. Likewise, tissues such as bone or cartilage can be associated with the composites prior to implantation within a patient. Examples of such cells include, but are not limited to, bone cells (such as osteoclasts, osteoblasts, and osteocytes), blood cells, epithelial cells, neural cells (e.g., neurons, astrocytes, and oligodendrocytes), and dental cells (odontoblasts and ameloblasts). Seeded cells can be autogenic, allogenic, or xenogeneic. Seeded cells can be encapsulated or non-encapsulated.

Other agents or compounds that can be incorporated into the composite of the subject invention include acid mucopolysaccharides including, but not limited to, heparin, heparin sulfate, heparinoids, dermatan sulfate, pentosan polysulfate, chondroitin sulfate, hyaluronic acid, cellulose, agarose, chitin, dextran, carrageenin, linoleic acid, and allantoin.

Proteins and other biologically active compounds that can be incorporated into, or included as an additive within, a composition comprising the bioactive coatings of the invention of the present invention include, but are not limited to, collagen (including cross-linked collagen), fibronectin, laminin, elastin (including cross-linked elastin), osteopontin, osteonectin, bone sialoproteins (Bsp), alpha-2HS-glycoproteins, bone Gla-protein (Bgp), matrix Gla-protein, bone phosphoglycoprotein, bone phosphoprotein, bone proteoglycan, protolipids, bone morphogenetic protein, cartilage induction factor, platelet derived growth factor and skeletal growth factor, enzymes, or combinations and biologically active fragments thereof. Other proteins associated with other parts of human or other mammalian anatomy can be incorporated or included as an additive, include proteins associated with cartilage, such as chondrocalcining protein, proteins associated with dentin, such as phosphoryin, glycoproteins and other Gla proteins, or proteins associated with enamel, such as amelogenin and enamelin. Agents incorporated into the composition of the present invention may or may not facilitate or enhance osteoinduction. Adjuvants that diminish an immune response can also be used in conjunction with the composite of the subject invention.

In one embodiment, the biologically active agents or compounds can first be encapsulated into microcapsules, microspheres, microparticles, microfibers, reinforcing fibers and the like to facilitate mixing and achieving controlled, extended, delayed and/or sustained release and combined with the cells of the invention. Encapsulating the biologically active agent can also protect the agent against degradation during formation of the composite of the invention.

In another embodiment of the invention, the biologically active agent is controllably released into a subject when the composition of the invention is implanted into a subject, due to bioresorption relying on the time scale resulting from cellular remodeling. In one aspect, the composition may be used to replace an area of discontinuity in the tissue. The area of discontinuity can be the result of trauma, a disease, disorder, or condition, surgery, injury, etc.

The peptides useful in the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride, or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N— and/or C— blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for analogs of proteins. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The invention also includes a kit comprising the composition of the invention and an instructional material which describes administering or using the composition. In another embodiment, this kit comprises a (preferably sterile) solvent suitable for dissolving or suspending the composition of the invention prior to administering the composition. Optionally, at least one growth factor and/or antimicrobial agent may be included in the kit. The present invention should be construed to include kits for improving vascular flow, stimulating angiogenesis, and for bone and wound healing. The invention includes a kit comprising a stimulator of angiogenesis or a compound identified in the invention, a standard, and an instructional material which describes administering the inhibitor or a composition comprising the stimulator. This should be construed to include other embodiments of kits that are known to those skilled in the art, such as a kit comprising a standard and a (preferably sterile) solvent suitable for dissolving or suspending the composition of the invention prior to administering the compound to a cell or an animal. Preferably, the animal is a mammal. More preferably, the mammal is a human.

In accordance with the present invention, as described above or as discussed in the Examples below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al., 1989 Molecular Cloning—a Laboratory Manual, Cold Spring Harbor Press; Glover, (1985) DNA Cloning: a Practical Approach; Gait, (1984) Oligonucleotide Synthesis; Harlow et al., 1988 Antibodies—a Laboratory Manual, Cold Spring Harbor Press; Roe et al., 1996 DNA Isolation and Sequencing: Essential Techniques, John Wiley; and Ausubel et al., 1995 Current Protocols in Molecular Biology, Greene Publishing.

The present invention may be embodied in other specific forms without departing from the spirit or essential attributes thereof. This invention encompasses all combinations of the different aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

Additional techniques and methods useful for the practice of the invention can be found in U.S. patent application Ser. No. 11/313,188, U.S. patent application Ser. No. 11/800,086, U.S. Pat. App. Pub. US 2007/0270844 A1, U.S. patent application Ser. No. 11/339,781, U.S. patent application Ser. No. 11/361,906, and U.S. patent application Ser. No. 11/598,900

The examples provided throughout his application are non-inclusive unless otherwise stated. They include but are not limited to the recited examples.

### EXAMPLES

Introduction

Tissue engineering strategies for bone repair have evolved in recent years to provide alternative bone grafting materials to replace damaged or missing bone due to trauma or disease. Each year, more than 500,000 bone graft procedures are performed in the United States and more than 2.2 million occur worldwide. Although great strides have been taken to create suitable bone substitute materials, 30 to 60% have failed upon implantation due to poor integration with the host tissue, and approximately 25% of cases of allograft implantation have been reported to be unsuccessful after 20 years in the recipient.

In order for bone or other tissues to heal effectively, an adequate microvascular network must be provided at the site of injury to deliver oxygen and nutrients to the resident tissue. The novel bioactive coating proposed will utilize S1P-receptor targeted drugs, specifically sphingosine-1-phosphate (S1P) or fingolimod (FTY720), encapsulated in poly(lactic-co-glycolic acid) (PLAGA) or polyhydroxybutyrate-co-valerate (PHBV), to promote angiogenesis and bone healing. Although several scientific studies conclude that FTY720 inhibits angiogenesis in tumor tissue, preliminary studies in our lab show that FTY720 significantly enhances angiogenesis and promotes bone healing in regenerating tissues. This product uses drug-loaded polymers to remodel the microvessel network and promote bone healing. The bioactive coating in the form of a polymer-coated allograft loaded with drug will serve as an effective bone graft substitute.

### Example 1

The present invention, which encompasses a novel bioactive coating to improve allograft incorporation”, includes the bioactive coating itself and the final polymer-coated bone allograft. To that end, a sphingosine 1-phosphate (S1P) receptor-targeted drug is encapsulated in a biocompatible, biodegradable polymer coating on a bone allograft. This drug-delivering allograft is useful for optimizing and localizing drug release kinetics, thus enhancing the wound and bone healing processes and the viability of bone allografts. In one aspect, two S1P receptor-targeted drugs, S1P and Fingolimod (FTY720), possess significant angiogenic properties, and either one can be incorporated into the polymer coating to alter the bone healing process. Either poly(D,L-lactic-co-glycolic acid) (PLAGA) or poly(3-hydroxybutrate-co-3-hydroxyvalerate) (PHBV) can be used as the biocompatible, biodegradable polymer coating.

Materials and Methods

Polymer Coating Solution

Polymer coatings using 50:50 or 85:15 poly-lactic-co-glycolic acid (PLAGA, Mw=72.3 kDa, Mw=123.6 kDa respectively) were purchased from Lakeshore Biomaterials, Birmingham, Ala. The polymer was dissolved in methylene chloride (MeCl) at three different wt./vol. ratios to achieve a range of coating viscosities (1:10, 1:12, 1:14). The solutions were agitated overnight to ensure homogeneity. For 50:50 PLAGA-coated, FTY720-loaded allografts (coated-loaded, C/L), 50:50 PLAGA was dissolved in MeCl at 1:12 (wt./vol.). FTY720 was then loaded into the polymer solution at 1:200 (wt./wt.). The solution containing polymer and drug was agitated briefly over heat until the drug dissolved completely. Samples were stored in a 4° C. vacuum desiccator for at least 24 hours to remove residual solvent and preserve drug activity.

Bone Allograft Preparation

Samples of tibial and femoral bone were harvested from Sprague Dawley retired breeders. Both tibias and femurs had soft tissue cleaned off, distal ends bone removed, and bone marrow flushed from the cavity. Remaining segments were agitated in a chloroform solution overnight to remove any residual fatty tissue. Following a brief 70% ethanol rinse, the allograft tissue was autoclaved for sterilization (121° C. at 15 PSI for 20 min) and allowed to fully dry. For polymer characterization studies, allograft samples were cut to 10 mm in length. For in vivo tibial defect studies, allograft samples were cut to 8 mm in length. Samples were stored in a −20° C. freezer until use. To coat allograft samples, the specified polymer solution was drawn up into the syringe containing the bone held in place; this allowed the solution to pass over and through the stationary sample. The solution was then expelled and this pumping motion continued for a total coating time of 10 min. Following coating, samples were stored in a −20° C. freezer for 72 hours to allow the slow evaporation of solvent.

Encapsulation Efficiency and Sphingolipid Release

Allograft samples were coated similarly to the above protocol with radio-labeled sphingosine-1-phosphate (S1P, Cayman Chemical, Ann Arbor Mich.) to measure drug release over a 14-day period. Specifically, 2.92 mg of S1P was resolubilized in MeCl to a final concentration of 2 mg/mL by frequent heating and vortexing. Using a conversion factor of 2.2×10⁶ cpm/μCi and 1 μCi/μL, 6.8 μL of Phosphorus-33 (³³P) (Perkin Elmer, Inc., Waltham, Mass.) was added to the S1P solution. Next, 583 mg of 50:50 PLAGA was added to the S1P-³³P solution to achieve a 1:12 (wt./vol.) polymer-solvent ratio. The final solution had 2.92 mg of S1P, 6.8 μL of ³³P, and 583 mg of 50:50 PLAGA mixed in 7 mL of MeCl. The coated allografts were then placed into a glass scintillation vial containing 50 μL of simulated body fluid (SBF) with 4% fatty acid free (FAF) BSA. Following 24 hours incubation, the allografts were removed from the vials and the remaining 4% FAF-BSA SBF was mixed with 5 mL of EcoScintA biodegradable scintillation solution for quantifying drug release using the Beckman Coulter liquid scintillation counter. Allografts were then placed into a new glass vial containing fresh 4% FAF-BSA SBF. This cycle was repeated for a 14-day period to achieve cumulative measurements. Encapsulation efficiency (maximum amount of S1P that can be released) was quantified by placing allografts in MeCl to completely dissolve the drug-containing polymer solution.

Critically-Sized Tibial Defect Model

All animal surgeries were performed according to an approved protocol from the UVA Animal Care and Use Committee. Briefly, adult male Sprague Dawley rats (˜400 g) were randomly assigned to three different experimental groups: uncoated allograft (U), coated 1:12 PLAGA (C), and coated 1:12 PLAGA loaded with 1:200 FTY720 (C/L). Animals were anesthetized with isoflurane gas. Following anesthetization, the dorsal skin was sterilized with betadine and 70% ethanol. A small incision was made longitudinally over the midshaft of the tibia. Subperiosteal dissection was performed once the subcutaneous tissue was dissected over the anterior aspect of the tibia. A Dremel rotary tool with a Diamond Wheel accessory was used to make an 8 mm defect slightly distal to the tibial tubercle and the 8 mm allograft segment was inserted into the defect. A stab incision was made just medial to the patellar tendon and a 19.5 gauge needle was inserted into the intramedullary canal, through the allograft, into the distal intramedullary canal to resistance, and tamped flush to the bone. The incision was irrigated and closed with 4-0 Nylon suture. Following closure, Buprenorphine was administered intramuscularly (0.1 mg/kg) after surgery and then as needed to minimize pain post surgery. Only the left tibia per animal was used, leaving the right side for normal function.

MicroCT Imaging (Polymer Coating Characterization, In Vivo, Ex Vivo)

To characterize polymer coating thickness and changes in allograft pore structure, samples were imaged using the quantitative micro-computed tomography (microCT) scanner (Scanco, Bassersdorf, Switzerland). Allograft samples from each group (PLAGA 1:10, PLAGA 1:12, PLAGA 1:14) were imaged utilizing a high-resolution 45 kVp scan. Following reconstruction of the 2D slices, an appropriate threshold matching the original grayscale image was chosen, contour lines were drawn around the allograft, and 3D images were generated. Total volume of bone and polymer, average pore size, and thickness of the polymer coating on the outer surface and inner canal of the bone were measured utilizing the 3D evaluation software. Subsequently, the polymer-coated allografts were agitated in MeCl overnight to dissolve the polymer coating and generate the uncoated bone sample.

The samples were re-imaged and analyzed using microCT to measure the bone volume only and the average pore size of the uncoated bone. Bone volume of the uncoated allograft was subtracted from total bone and polymer volume of the coated allograft to obtain a measure of polymer volume. Average pore size of the uncoated allograft was subtracted from the average pore size of the coated allograft to obtain a measure of the change in pore size after coating; the resulting number was negative if the average pore size decreased after coating. The thresholds (bone: lower=200, upper=1000; polymer: lower=112, upper=200; bone+polymer: lower=112, upper=1000) and scan parameters (bone: support=4, width=1.2; polymer: support=6, width=3.4; bone+polymer: support=6, width=1.2) were kept constant throughout the entire study.

New bone healing along the length of the allograft insertion site was imaged post-operatively at 0, 2, 4, and 6 weeks. Animals were anesthetized by isofluorane gas and imaged for 11.7 minutes utilizing a low-resolution 45 kVp scan. At end time points, animals were euthanized, the hindlimb was disarticulated, the soft tissue was stripped, and the intramedullary pin was removed. Ex vivo scans of each specimen were obtained utilizing a high-resolution 45 kVp scan. Following reconstruction of the 2D slices, an appropriate threshold matching the original grayscale images was chosen. Contour lines were drawn to appropriately select a standard window of 2 mm×0.15 mm drawn 0.4 mm away from the interface between the host bone and allograft. These standard contour lines were drawn around both the host bone and allograft but excluded the hollow canal. 3D images were created from 2D slices, and the bone density within the contour lines was calculated using the 3D evaluation program. The standard window, threshold (260 for high resolution ex vivo bone volume data, 200 for low resolution in vivo qualitative 3D images), and scan parameters (support=4, width=1.2) were kept constant throughout the entire study.

Evaluation of Post-operative Mechanical Properties

Excised tibias were tested under compression in an Instron 4511 machine to determine the elastic modulus and ultimate compressive strength (UCS) of uncoated, coated, and coated/loaded (50:50 PLAGA+1:200 FTY720) allograft samples. The primary goal was to determine the strength of the integrated structure (host-bone-allograft) 6 weeks post-op. All compression testing was performed at a rate of 1 mm/min until sample failure.

Evaluation of Post-Operative Tissue Histology

Following ex vivo microCT scanning at 6 weeks, tibia samples were fixed in 10% formalin, decalcified using Richard Scientific Decalcifying Solution (Kalamazoo, Mich.) for 2 days at room temperature, and dehydrated overnight. Half the samples were embedded in paraffin and the other half cryo-sectioned. Each sample was cut along the sagittal plane at the midline of the defect. For smooth muscle α-actin (SMA) staining to visualize mature vessel lumens, four 7 μm-thick histological sections per sample (paraffin-embedded) were dewaxed, rehydrated, blocked 3×10 minutes in PBS/saponin/BSA and immunolabeled for SMA using CY3-conjugated monoclonal anti-SMA (Sigma Aldrich) diluted 1:200 in PBS/saponin/BSA. Slides were incubated with antibodies for approximately 15 hours at 4° C. Subsequently, tissues were washed 3 times in PBS/saponin for 10 min each. Samples were then mounted using a 50:50 solution of PBS and glycerol.

For CD45 staining to visualize leukocyte recruitment, four 7 μm-thick histological sections per sample (cryo-embedded) were blocked 3×10 minutes in PBS/saponin/BSA and immunolabeled with CD45 (BD Pharmingen) diluted 1:50 in PBS/saponin/BSA. Slides were incubated with antibodies for approximately 15 hours at 4° C. followed by the secondary antibody streptavidin 488 for additional 15 hours at 4° C. Subsequently, tissues were washed 3 times in PBS/saponin for 10 min each. Samples were then mounted using a 50:50 solution of PBS and glycerol. For hematoxylin and eosin (H&E) and Masson's Trichrome staining to visualize collagen fiber morphology, four 7 μm-thick histological sections per sample (paraffin-embedded) were dewaxed, rehydrated, and appropriately stained. Immunostained sections were imaged using both a Nikon TE 2000-E2 confocal microscope and Zeiss Axioskop 40 inverted microscope. Representative images were acquired using 4×, 10×, and 50× objectives. To quantify changes in vascular remodeling (particularly recruitment of mural cells) in response to FTY720, SMA-positive cells that formed an obvious lumen were quantified in each tissue section. Total tissue area was measured in ImageJ, and the FTY720-mediated response was represented as a fraction of SMA-positive lumens per total tissue area.

Statistical Significance

Results are presented as mean±SEM. Statistical analysis of polymer coating thickness, bone density, mechanical properties, and vessel density was performed using a one-way General Linear ANOVA, followed by Tukey's test for pairwise comparisons. Significance was asserted at p<0.05.

Results—

Polymer-Coated Allograft Characterization

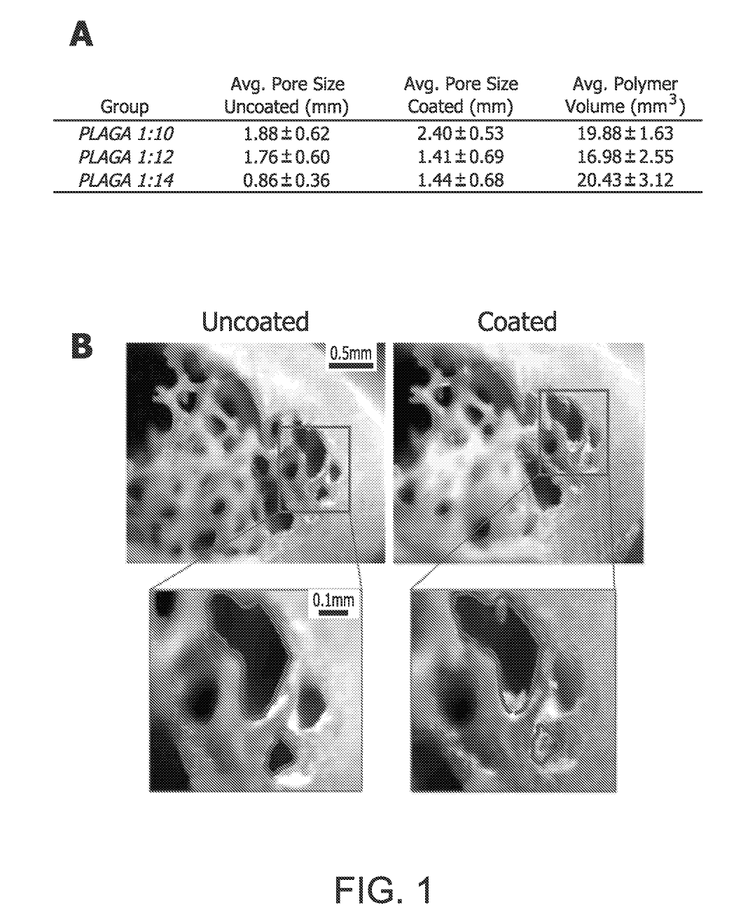

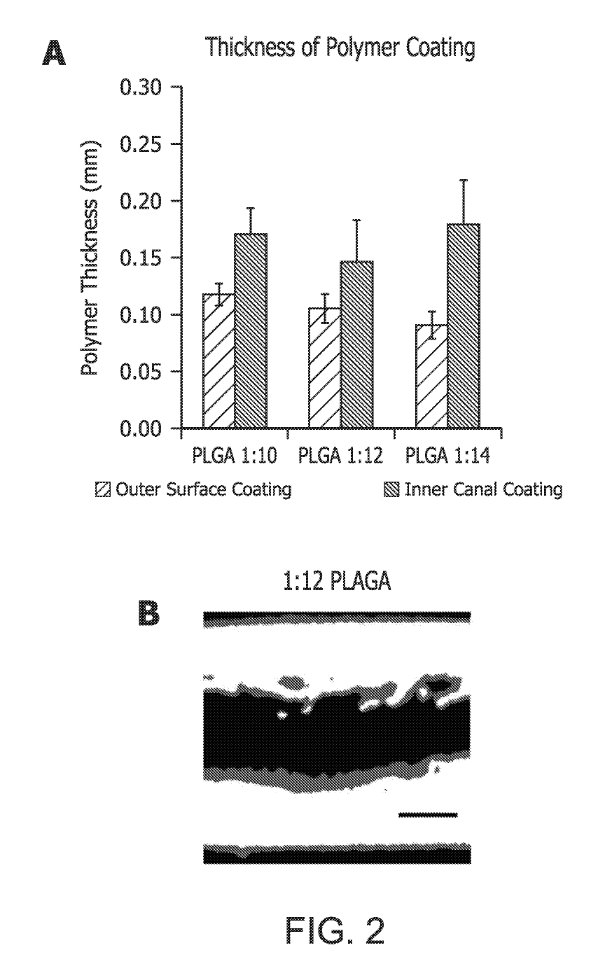

Changes in average pore size between coated and uncoated samples were measured by microCT imaging analysis. Allografts were scanned before and after coating with each PLAGA concentration (1:10, 1:12, 1:14) (FIG. 1A). As expected, data trends showed average pore size decreases following polymer coating. Despite decreases in average pore size, macroscopic cross-sections of tibial bone revealed preservation of many larger pore features following polymer coating (FIG. 1B). These images suggested that although average pore size was reduced in most groups following coating, the overall pore structure was maintained; this property is critical for osteoconductivity and fracture healing. Furthermore, the total volume of polymer implanted at the defect site was less than 5% of the allograft itself, thus keeping degradation byproducts to a minimum. The thickness of polymer coating on both inner and outer surfaces was also measured by microCT imaging. Thicknesses ranged from 0.1 mm to 0.2 mm, where smooth outer surfaces retained thinner layers of polymer compared with porous inner surfaces (FIG. 2A). Measurements of thicknesses were similar across polymer types and solution ratios. Representative images from each group display polymer coating in red, bone tissue in white, and marrow cavity in black (FIG. 2B).

Encapsulation Efficiency and Drug Release

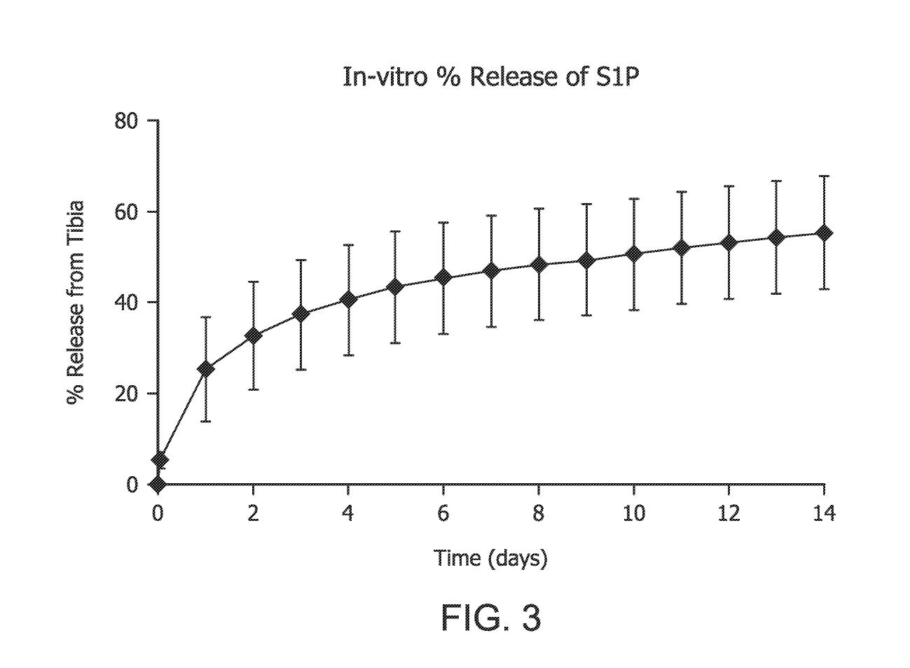

To determine total amount of drug loaded into allograft implants and the kinetics of drug release, in vitro experiments were conducted utilizing allografts loaded with S1P-³³P (FIG. 3). Complete degradation of PLAGA occurs between 6-8 weeks [20]. However, substantial S1P release was expected to occur earlier, given the bulk degradation profile of PLAGA. We found approximately 0.64 mg of the original 1 mg of S1P was actually loaded within the polymer-coated allograft (64% loading efficiency). After 14 days incubation, 0.57 mg of S1P was detected in the simulated body fluid (SBF). This amount was assumed to be the total amount of S1P loaded given the minimal increases in S1P release at later time points. An initial burst release of drug was observed during the first five days, typical for 50:50 PLAGA degradation profiles.

In Vivo MicroCT Analysis

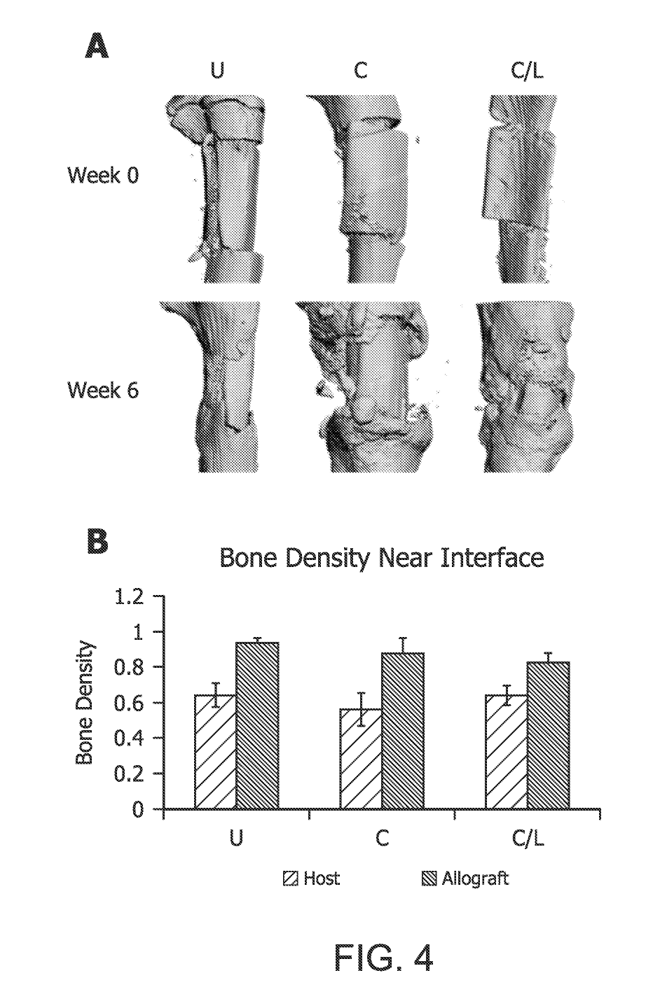

Bone healing was monitored at 2, 4, and 6 weeks post surgery utilizing low-resolution in vivo microCT imaging. At six-week endpoints, samples were scanned ex vivo at high resolution (FIG. 4A) and quantitative values of bone density near the host bone-allograft interface were calculated for each sample from the high resolution ex vivo scans (FIG. 4B). Qualitative images suggest better integration of remodeled bone along the length of the implanted allograft in the coated-loaded (C/L) group compared with unloaded (U) and coated (C) groups. This qualitative analysis suggests a positive effect of FTY720 on the spatial distribution and integration of new bone at the implant-tibial interface. Additionally, bone density measurements were calculated for high resolution ex vivo scans for all groups. Comparisons between bone density in host tissue and allograft regions suggest a closer match of densities within the C/L group compared with U and C groups. Thus, FTY720 treatment may promote osseous tissue remodeling such that allograft/bone density interface is well-matched to promote long-term allograft incorporation.

### Mechanical Testing

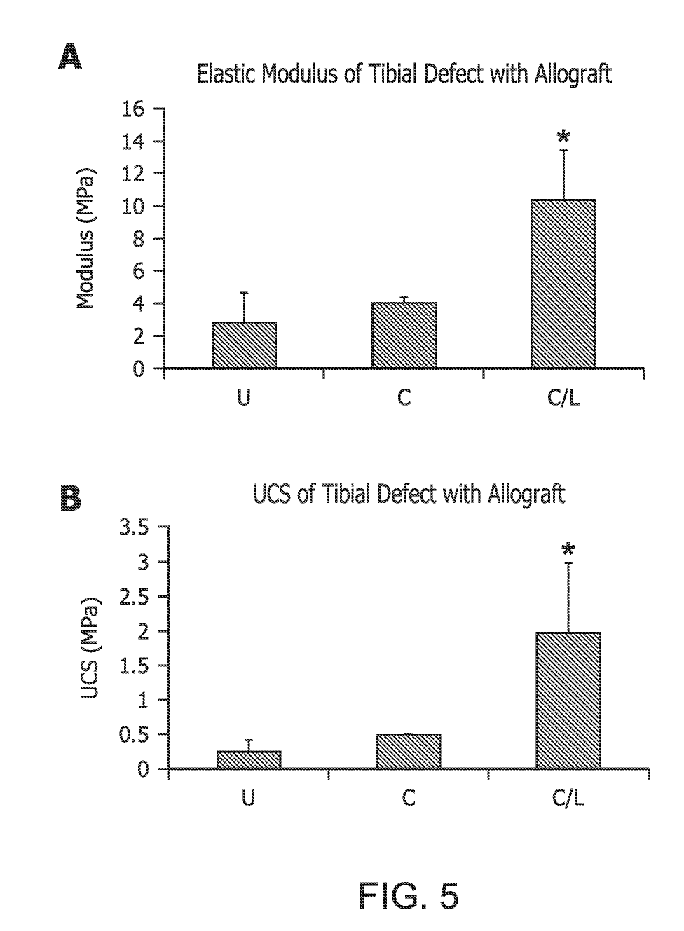

A leading cause of allograft failure or post-operative complications includes mechanical instability at the bone-allograft interface. Thus, mechanical testing following six-weeks healing was performed using an Instron machine to determine the elastic modulus of all groups (U, C, C/L) ex vivo (FIG. 5A). To determine the elastic modulus, the slope was calculated between the values of 0.07 to 0.08 strain, since this was the interval in which the elastic region existed in all the graphs. Both values of elastic modulus and ultimate compressive strength (FIG. 5B) were significantly higher in C/L groups compared with both U and C groups. Superior mechanical properties in C/L groups supported qualitative microCT images and quantitative bone density measurements, suggesting that local FTY720 delivery may effectively increase the structural integrity of the allograft-host bone interface.

Immunofluorescence Analysis

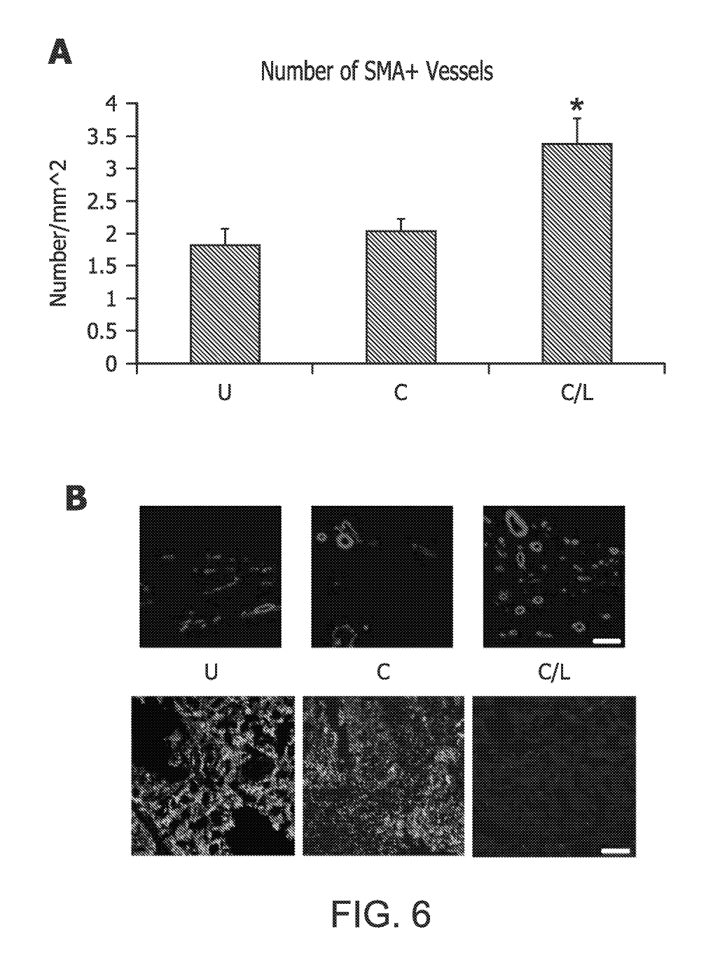

Previous observations demonstrated significant increases in smooth muscle cells investment when FTY720 was delivered locally in both cranial defect and dorsal skinfold window chamber models [18,19]. In massive allograft implants, poor vascularization is a leading cause for post-operative complications and failure of massive bone allografts. To this end, FTY720 was locally delivered to encourage vascularization of the interface region and subsequently smooth muscle cell was quantified. Similar to previous applications, FTY720 treatment significantly increased the number of SMCs within the interface regions compared with C and U groups (FIG. 6A). Immunostaining showed continuous lumens with signature “tire-track” alpha-smooth muscle actin staining within allograft regions (FIG. 6B). In additional to enhancing bone and vascular remodeling, FTY720 treatment in vivo has also demonstrated immunosuppressive properties. Previously, we have shown decreased monocyte recruitment in cranial defects following FTY720 treatment [19]. Here, using a pan-hematopoietic stain, qualitative images suggested reductions in the number of leukocytes within the allograft-host bone interface when treated with FTY720 (FIG. 6C). Reductions in immune cell trafficking to the wound site were consistent with previous results in cranial defect and dorsal skinfold window chamber tissues.

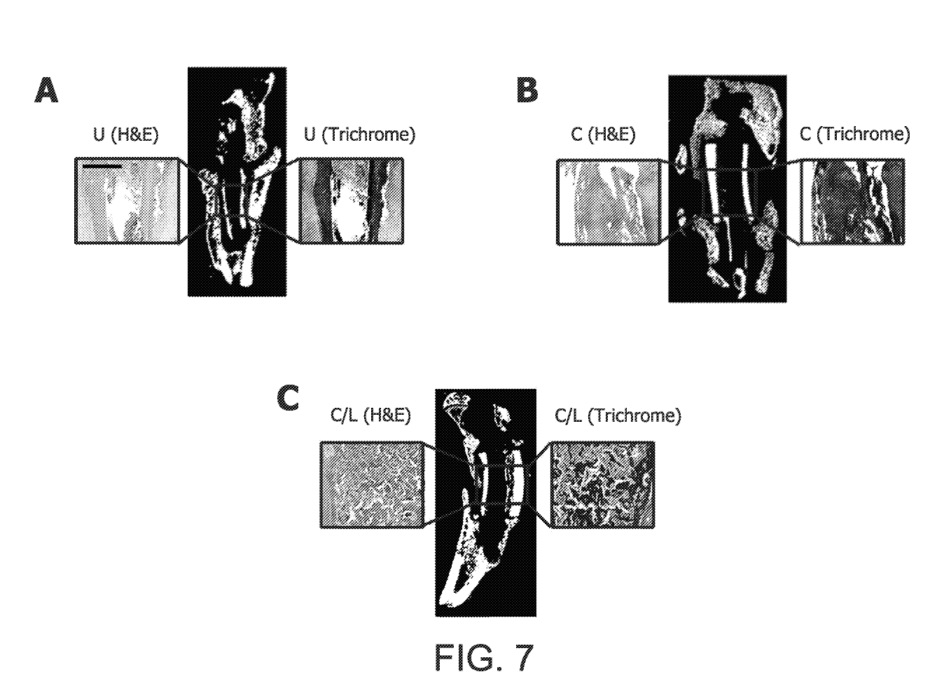

Bone remodeling and collagen fibril alignment were visualized through H&E and Masson's trichrome staining of longitudinal cross-sections of allograft-host bone interfaces. FTY720 treated groups (FIG. 7C) showed superior collagen alignment, osseous tissue generation along the outer edge of the allograft tissue, better preservation of inner cancellous porous regions, and preservation of allograft-tibial alignment compared with U (FIG. 7A) and C (FIG. 7B) groups.

Discussion

Poor vascularization and hampered osseous graft integration are commonly associated with long-term complication and poor functional outcome of massive skeletal allografts [3-5, 8, 9]. Promising new strategies designed to directly address limitations in vascularization of allograft regions include co-delivery of stem cells [21], platelet-rich plasma [22], recombinant proteins [23], and gene therapy [24]. In particular, recent clinical data suggests that rhBMP2 proteins can be effective in improving repair success in non-union fracture healing and cervical spine fusion; however, concerns persist regarding the delivery of large amounts of rhBMP2 and associated complications, including edema and ectopic bone formation [25,26]. Moreover, adjunct therapies focused exclusively on enhancement of overall bone mass to aid graft incorporation have failed to significantly reduce these post-operative complications [7].

In this study, we have coated devitalized tibial grafts with a thin layer of PLAGA to support the controlled delivery of FTY720 to the host-graft interface, while successfully maintaining overall porous structure of cancellous bone.

Utilizing biodegradable polymer systems is an effective strategy to incorporate sustained drug release in vivo. Previous factor-eluting polymer coating systems have utilized titanium implants coated with poly(D,L-lactide) to locally deliver TGF-beta-1 and IGF-1 [27]. In these studies, enhanced mechanical fixation and osseointegration were observed at the host-graft interface and minimal fibrous scarring was noted. Others have evaluated the effectiveness of coating cortical bone with polypropylene fumarate) foam to enhance allograft incorporation [27-29]. In these studies, strength to failure of the coated allograft groups was stronger at the interface compared with uncoated grafts. Additionally, authors suggested that polymer-coated grafts were better protected from excessive osteoclastic resorption, a process that can often enhance fibrous scar invasion and ultimately deteriorate effective allograft incorporation. To this end, we developed a novel polymer coating system, where a continuous polymer layer is coated across the entire porous allograft surface, creating a sustainable localized delivery mechanism within a massive tibial defect site. Using this approach, we can capitalize on the existing properties of devitalized bone and provide a barrier from excessive osteoclastic resorption. Moreover, local administration of FTY720 allows us to exploit the multiple biological functions of the S1P receptor-signaling axis to promote bone healing.

Fundamentally, we understand that following injury, local vasculature and damaged osseous tissue incite an inflammatory response that proceeds via complement activation, recruitment of monocytes/macrophages, and eventual clearance of damaged tissues. Additionally, timely integration of functional vascular networks is critical to initiate appropriate osseous remodeling, similar to the microvessels in developing embryos, which serve as a bed for osteoblastic development and differentiation [30, 31]. Given the emerging evidence elucidating downstream effects of selective S1P1/S1P3 activation on microvascular development and stabilization, we believe that the delivery of FTY720 may be effective for inducing growth and development of mature microvascular networks within allograft regions, leading to improved bone healing outcomes and reducing post-op complications. Specifically, our results show that local delivery of FTY720 led to two significant outcomes. First, it enhanced the number of smooth-muscle-invested vessels within allograft tissue sections, a hallmark of mature microvessel network growth. Second, it promoted significant new bone formation with statistically significant increases in compressive modulus and ultimate tensile strength after only 6 weeks implantation.

These results are consistent with the wealth of evidence supporting the critical role of S1P receptors in vascular development and signaling in both vascular endothelial cells and smooth muscle cells. Global knockout of S1P1 is embryonic lethal through hemorrhaging as a result of aberrant recruitment of smooth muscle cells to nascent endothelial tubes [32]. Tissue-specific knockout of S1P1 in endothelial cells phenocopies the global S1P−/− knockout phenotype, likely due to disruption of N-cadherin-dependent adhesive contacts between endothelial cells and mural cells that are critical to vascular maturation in the embryo [33]. Moreover, in vitro and in vivo studies of mature SMCs show that S1P1 and S1P3 promote the proliferation and migration of SMCs, which are critical to vascular network maturation [17]. These results support the idea that pharmacological targeting of S1P1/S1P3 with drugs like FTY720 is an exciting new approach to therapeutic neovascularization and enhancement of bone healing.

Modulation of local immune response and foreign body reaction may also be part of the mechanism by which FTY720 promotes allograft incorporation. Clinically, systemic delivery of FTY720 has shown potent immunomodulatory effects, preventing lymphocyte egress and recirculation from the thymus and peripheral lymphoid organs [34]. Moreover, S1P1 antagonizes pathologic inflammation by preventing monocyte adhesion to activated endothelium [35], and recent evidence has shown that treatment with FTY720 ameliorates aosteoporosis in mice by reducing the osteoclast population size [15]. In our study, we observed significant reduction in the number of CD45+ leukocytes near graft sections. This result was consistent with previous studies from our group showing dramatic reduction in CD45+ cell recruitment in response to localized FTY720 release in both dorsal skinfold window chamber studies (data not shown) and bone defects studies [18]. As part of the innate immune response, fibrous tissue may often invade and surround bone implants, creating a discontinuous strain interface that is mechanically inferior to cortical bone. Moreover, the formation of fibrous tissue is a significant barrier to microvascularization and osseous tissue ingrowth. Thus, delivery of FTY720 may act locally to reduce the formation of fibrous tissue invasion of allograft implants by reducing leukocyte trafficking near graft regions.

Because such strong coordination exists between inflammatory cell recruitment and wound healing progression towards resolution, inflammation-driven modulation of S1P receptor signaling may also play an important role in recruitment of marrow derived mesenchymal progenitor cells to sites of tissue regeneration. For example, recent data indicate that the therapeutic success of transplanted progenitor or stem cells can be improved by pharmacological stimulation of surface receptors in order to enhance homing to sites of injury and ischemia. Indeed, several studies have explored the dynamics of SDF-1α/CXCR4 cross talk with S1P receptor signaling and its possible role in directing MPC adhesion and recruitment. Activation of S1P1 and S1P3 by FTY720 augments SDF-1-dependent transendothelial progenitor cell migration in vitro and bone marrow homing [36].

These observations support previous data from our laboratory suggesting that local delivery of FTY720 to cranial defects may enhance bone ingrowth through recruitment of local bone progenitor cells from the meningeal dura mater and adjacent soft tissues [19].

Conclusions

Allograft products continue to be an attractive option for replacement strategies in massive skeletal defects including spinal fusion and joint revision. However, devitalized grafts must be quickly re-populated and vascularized in vivo for proper long-term functional success. Here, we have successfully coated grafts with a thin layer of PLAGA, successfully maintained overall porous structure of cancellous bone, locally delivered FTY720 to the host-graft interface, enhanced mechanical stability and vascular recruitment, appropriately remodeled osseous tissue surrounding the interface, and reduced leukocyte trafficking near the implant site. Such results support continued evaluation of drug-eluting allografts as a viable strategy to improve functional outcome and long-term success of massive cortical allograft implants.

### BIBLIOGRAPHY

U.S. patent application Ser. No. 11/313,188
U.S. patent application Ser. No. 11/800,086
U.S. patent application Ser. No. 11/339,781
U.S. patent application Ser. No. 11/361,906
U.S. patent application Ser. No. 11/598,900
U.S. Pat. No. 7,241,790,
U.S. Pat. No. 7,560,477
U.S. Pat. No. 7,638,637
U.S. patent application Ser. No. 12/179,816
U.S. patent application Ser. No. 12/470,011
U.S. patent application Ser. No. 12/470,017
U.S. patent application Ser. No. 12/189,010
U.S. patent application Ser. No. 12/470,009
U.S. Pat. App. Pub. US 2007/0270844 A1
PCT Pat. App. WO US/2009/023854
1. Giannoudis P V, Dinopoulos H, Tsiridis E. Bone substitutes: an update. Injury 2005; 36 Suppl 3:S20-7.
2. Greenwald A S, Boden S D, Goldberg V M, Khan Y, Laurencin C T, Rosier R N, American Academy of Orthopaedic Surgeons. The Committee on Biological Implants. Bone-graft substitutes: facts, fictions, and applications. J Bone Joint Surg Am 2001; 83-A Suppl 2 Pt 2:98-103.
3. Wheeler D L, Enneking W F. Allograft bone decreases in strength in vivo over time. Clin Orthop Relat Res 2005; (435):36-42.
4. Delloye C, de Nayer P, Allington N, Munting E, Coutelier L, Vincent A. Massive bone allografts in large skeletal defects after tumor surgery: a clinical and microradiographic evaluation. Arch Orthop Trauma Surg 1988; 107:31-41.
5. Mankin H J, Hornicek F J, Raskin K A. Infection in massive bone allografts. Clin Orthop Relat Res 2005; (432):210-216.
6. Vander Griend R A. The effect of internal fixation on the healing of large allografts. J Bone Joint Surg Am 1994; 76:657-663.
7. Boraiah S, Paul O, Hawkes D, Wickham M, Lorich D G. Complications of recombinant human BMP-2 for treating complex tibial plateau fractures: a preliminary report. Clin Orthop Relat Res 2009; 467:3257-3262.
8. Delloye C, Cornu O, Druez V, Barbier O. Bone allografts: What they can offer and what they cannot. J Bone Joint Surg Br 2007; 89:574-579.
9. Thompson R C, Jr, Pickvance E A, Garry D. Fractures in large-segment allografts. J Bone Joint Surg Am 1993; 75:1663-1673.
10. Zhang H, et al., Sphingosine-1-phosphate, a novel lipid, involved in cellular proliferation. J Cell Biol 1991; 114:155-167.
11. Cuvillier O, Pirianov G, Kleuser B, Vanek P G, Coso O A, Gutkind S, et al. Suppression of ceramide-mediated programmed cell death by sphingosine-1-phosphate. Nature 1996; 381:800-803.
12. Lee M J, Thangada S, Paik J H, Sapkota G P, Ancellin N, Chae S S, et al. Akt-mediated phosphorylation of the G protein-coupled receptor EDG-1 is required for endothelial cell chemotaxis. Mol Cell 2001; 8:693-704.
13. Ryu J, Kim H J, Chang E J, Huang H, Banno Y, Kim H H. Sphingosine 1-phosphate as a regulator of osteoclast differentiation and osteoclast-osteoblast coupling. EMBO J. 2006; 25:5840-5851.
14. Pebay A, Bonder C S, Pitson S M. Stem cell regulation by lysophospholipids. Prostaglandins Other Lipid Mediat 2007; 84:83-97.
15. Ishii M, Egen J G, Klauschen F, Meier-Schellersheim M, Saeki Y, Vacher J, et al. Sphingosine-1-phosphate mobilizes osteoclast precursors and regulates bone homeostasis. Nature 2009; 458:524-528.
16. Walter D H, Rochwalsky U, Reinhold J, Seeger F, Aicher A, Urbich C, et al. Sphingosine-1-phosphate stimulates the functional capacity of progenitor cells by activation of the CXCR4-dependent signaling pathway via the S1P3 receptor. Arterioscler Thromb Vasc Biol 2007; 27:275-282.
17. Wamhoff B R, Lynch K R, Macdonald T L, Owens G K. Sphingosine-1-phosphate receptor subtypes differentially regulate smooth muscle cell phenotype. Arterioscler Thromb Vasc Biol 2008; 28:1454-1461.
18. Sefcik L S, Petrie Aronin C E, Wieghaus K A, Botchwey E A. Sustained release of sphingosine 1-phosphate for therapeutic arteriogenesis and bone tissue engineering. Biomaterials 2008; 29:2869-2877; ePub on Apr. 11, 2008.
19. Petrie Aronin C, Sefcik L S, Tholpady A, Sadik K W, Tholpady S S, Macdonald T L, et al. FTY720 promotes local microvascular network formation and regeneration of cranial bone defects. Tissue Eng Part A 2009.
20. Ramchandani M, Pankaskie M, Robinson D. The influence of manufacturing procedure on the degradation of poly(lactide-co-glycolide) 85:15 and 50:50 implants. Controlled Release 1997; 43:161-173.

21. Lucarelli E, Fini M, Beccheroni A, Giavaresi G, Di Bella C, Aldini N N, et al. Stromal stem cells and platelet-rich plasma improve bone allograft integration. Clin Orthop Relat Res 2005; (435):62-68.
22. Han B, Woodell-May J, Ponticiello M, Yang Z, Nimni M. The effect of thrombin activation of platelet-rich plasma on demineralized bone matrix osteoinductivity. J Bone Joint Surg Am 2009; 91:1459-1470.
23. Nevins M, Hanratty J, Lynch S E. Clinical results using recombinant human platelet-derived growth factor and mineralized freeze-dried bone allograft in periodontal defects. Int J Periodontics Restorative Dent 2007; 27:421-427.
24. Yazici C, Yanoso L, Xie C, Reynolds D G, Samulski R J, Samulski J, et al. The effect of surface demineralization of cortical bone allograft on the properties of recombinant adeno-associated virus coatings. Biomaterials 2008; 29:3882-3887.
25. Cahill K S, Chi J H, Day A, Claus E B. Prevalence, complications, and hospital charges associated with use of bone-morphogenetic proteins in spinal fusion procedures. JAMA 2009; 302:58-66.
26. Shields L B, et al. Adverse effects associated with high-dose recombinant human bone morphogenetic protein-2 use in anterior cervical spine fusion. Spine (Phila Pa 1976) 2006; 31:542-547.
27. Lamberg A, Schmidmaier G, Soballe K, Elmengaard B. Locally delivered TGF-beta1 and IGF-1 enhance the fixation of titanium implants: a study in dogs. Acta Orthop 2006; 77:799-805.
28. Lewandrowski K U, Bondre S, Hile D D, Thompson B M, Wise D L, Tomford W W, et al. Porous poly(propylene fumarate) foam coating of orthotopic cortical bone grafts for improved osteoconduction. Tissue Eng 2002; 8:1017-1027.
29. Bondre S, Lewandrowski K U, Hasirci V, Cattaneo M V, Gresser J D, Wise D L, et al. Biodegradable foam coating of cortical allografts. Tissue Eng 2000; 6:217-227.
30. Streeten E A, Brandi M L. Biology of bone endothelial cells. Bone Miner 1990; 10:85-94.
31. Drushel R F, Pechak D G, Caplan A I. The anatomy, ultrastructure and fluid dynamics of the developing vasculature of the embryonic chick wing bud. Cell Differ 1985; 16:13-28.
32. Liu Y, Wada R, Yamashita T, Mi Y, Deng C X, Hobson J P, et al. Edg-1, the G protein-coupled receptor for sphingosine-1-phosphate, is essential for vascular maturation. J Clin Invest 2000; 106:951-961.
33. Paik J H, Skoura A, Chae S S, Cowan A E, Han D K, Proia R L, et al. Sphingosine 1-phosphate receptor regulation of N-cadherin mediates vascular stabilization. Genes Dev 2004; 18:2392-2403.
34. Matloubian M, Lo C G, Cinamon G, Lesneski M J, Xu Y, Brinkmann V, et al. Lymphocyte egress from thymus and peripheral lymphoid organs is dependent on S1P receptor 1. Nature 2004; 427:355-360.
35. Whetzel A M, et al. Sphingosine-1 phosphate prevents monocyte/endothelial interactions in type 1 diabetic NOD mice through activation of the S1P1 receptor. Circ Res 2006; 99:731-739.
36. Kimura T, et al. The sphingosine 1-phosphate receptor agonist FTY720 supports CXCR4-dependent migration and bone marrow homing of human CD34+ progenitor cells. Blood 2004; 103:4478-4486.
37. Kawanabe et al., Sphingosine 1-phosphate accelerates wound healing in diabetic mice. Dermatol. Sci. 2007, 48:1:53-60. Epub. 2007 Jul. 20

The disclosures of each and every patent, patent application, and publication cited herein are hereby incorporated by reference herein in their entirety. One of skill in the art will appreciate that the superiority of the compositions and methods of the invention relative to the compositions and methods of the prior art are unrelated to the physiological accuracy of the theory explaining the superior results.

Headings are included herein for reference and to aid in locating certain sections. These headings are not intended to limit the scope of the concepts described therein under, and these concepts may have applicability in other sections throughout the entire specification.

Other methods which were used but not described herein are well known and within the competence of one of ordinary skill in the art of clinical, chemical, cellular, histochemical, biochemical, molecular biology, microbiology and recombinant DNA techniques.

The description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

What is claimed is:

1. A method of enhancing bone healing in a subject in need thereof, said method comprising administering to said subject a composition comprising a biocompatible polymer and an effective amount of S1P, or a composition comprising a biocompatible polymer and an effective amount of a compound having S1P receptor selective agonist or antagonist activity or a biologically active derivative or analog thereof, thereby enhancing bone healing.

2. The method of claim 1, wherein said bone healing is healing of a bone allograft.

3. The method of claim 1, wherein said compound having S1P agonist activity is selected from the group consisting of: 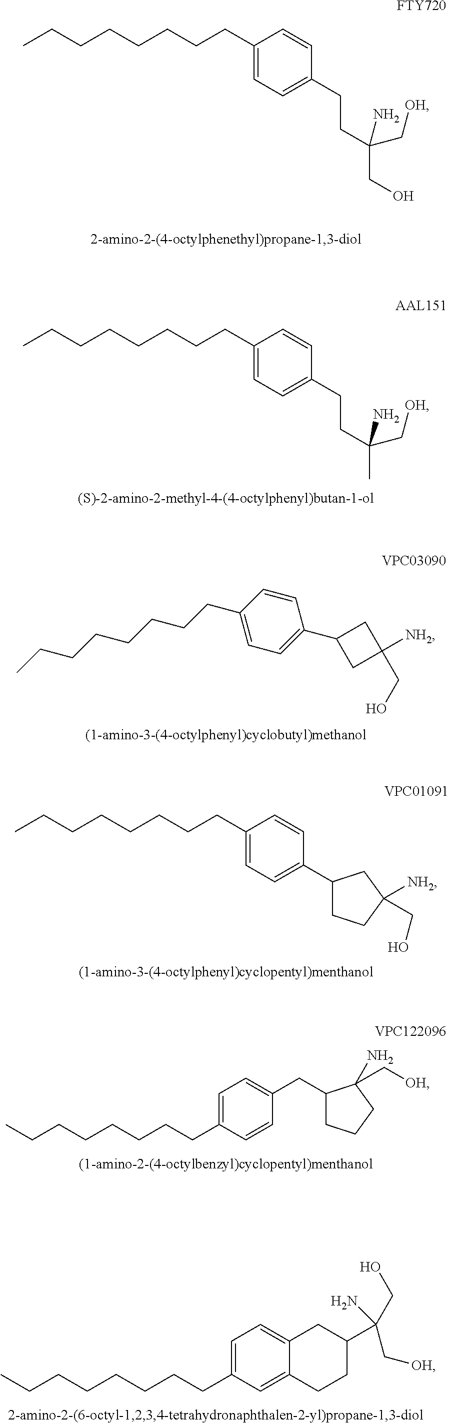 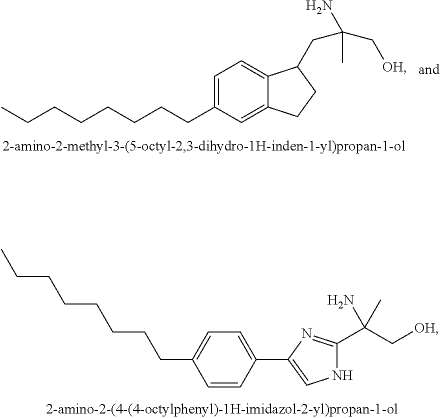
or a biologically active derivative or analog thereof.

4. The method of claim 2, wherein said polymer is PLAGA or PHBV.

5. The method of claim 4, wherein said composition is coated on a bone allograft and said bone allograft is administered to said subject.

6. The method of claim 4, wherein said PLAGA is a 50:50 or 85:15 mixture of the 72.3 kDa and 123.6 kDa forms.

7. The method of claim 2, wherein said PLAGA is mixed with methylene chloride to form a PLAGA:methylene chloride solution.

8. The method of claim 7, wherein PLAGA is mixed with methylene chloride at weight to volume ratios of 1:10, 1:12, or 1:14.

9. The method of claim 8, further wherein FTY720 or a biologically active derivative or analog thereof is added to said PLAGA:methylene chloride solution.

10. The method of claim 9, wherein said FTY720 or a biologically active derivative or analog thereof is added to said PLAGA:methylene chloride solution at a ratio of about 1:200 weight:weight.

11. The method of claim 1, wherein said composition further comprises at least one purified antimicrobial agent.

12. The method of claim 1, wherein said composition is administered to said subject using a method selected from the group consisting of directly, topically, subcutaneously, and parenterally.

13. The method of claim 12, wherein said composition is administered directly.

14. The method of claim 1, wherein said method enhances angiogenesis.

15. The method of claim 1, wherein said subject is human.

16. The method of claim 1, wherein said method increases the structural integrity of a bone allograft-host bone interface.

17. A method of treating a wound in a subject in need thereof, said method comprising administering a composition of claim 1 to said wound, thereby treating a wound.

18. The method of claim 17, wherein said wound is a bone wound.

19. The method of claim 18, wherein said method enhances bone healing.

20. A kit for administering a composition of the invention for treating a wound or for enhancing bone healing, said kit comprising a composition comprising a biologically compatible polymer and at least one S1P receptor selective agonist or antagonist, optionally a pharmaceutically acceptable carrier, optionally at least one antimicrobial agent, optionally at least one additional therapeutic agent, an applicator, and an instructional material for the use thereof.
